(19) United States
(12) Patent Application Publication
(10) Pub. No.: US 2021/0139528 A1
(43) Pub. Date: May 13, 2021

(54) CRYSTALLINE FORMS OF OBETICHOLIC ACID

(71) Applicant: INTERCEPT PHARMACEUTICALS, INC., New York, NY (US)

(72) Inventors: Gabriel M. GALVIN, San Diego, CA (US); Melissa REWOLINSKI, San Diego, CA (US)

(21) Appl. No.: 16/491,798

(22) PCT Filed: Mar. 7, 2018
(86) PCT No.: PCT/US18/21307

§ 371 Date: Sep. 6, 2019

Related U.S. Application Data
(60) Provisional application No. 62/468,592, filed on Mar. 8, 2017.

(51) Int. Cl. C07J 9/00

(52) U.S. Cl. CPC C07J 9/005; C07B 2200/13

(57) ABSTRACT

The crystalline forms of obeticholic acid and methods of preparation and use thereof are described.

### BACKGROUND

The variety of possible solid forms creates potential diversity in physical and chemical properties for a given pharmaceutical compound. The discovery and selection of solid forms are of great importance in the development of an effective, stable and marketable pharmaceutical product. Although active pharmaceutical ingredients (APIs) can be part of a medicine in amorphous form, the major part of medicines contains APIs in crystalline forms like salts or polymorphs. The search for new crystalline forms, e.g., cocrystals, involves significant effort and presents a lot of interest, because new cocrystalline forms can further improve physicochemical and pharmaceutical properties of APIs.

Obeticholic acid (OCA), an FXR agonist, has been marketed since 2016 as the drug product OCALIVA® for the treatment of primary biliary cholangitis (PBC) in adult patients. OCA is indicated for the treatment of PBC in combination with Ursodeoxycholic acid (UDCA) in adults with an inadequate response to UDCA or as monotherapy in adults unable to tolerate UDCA. The effectiveness of OCALIVA® in these patients is based on a study that showed a reduction in the liver enzyme alkaline phosphatase (ALP). OCALIVA® has addressed a long-time unmet need in the treatment of PBC.

OCA has shown anti-cholestatic, anti-inflammatory, and anti-fibrotic effects mediated by FXR activation in preclinical and clinical studies. As such, there is a growing interest and need in further exploring new solid forms of OCA, e.g., crystalline and cocrystalline forms, which will lead to the development of new dosage forms permitting more convenient administration to patients including various patient populations, limited amount of impurities upon storage, suitable impurity profile to minimize potential toxicity, accurate delivery of intended dose, improved treatment regimens that maximize biologic activity, and other potential pharmaceutical advantages.

### SUMMARY

The present disclosure relates to crystalline forms of obeticholic acid including salts and cocrystals of obeticholic acid.

In one aspect, this disclosure pertains to cocrystalline forms of obeticholic acid (OCA) and a co-former

[chemical structure]

In some embodiments, the present disclosure pertains to the cocrystalline forms of OCA, wherein the co-former is a bile acid derivative.

In some embodiments, the bile acid derivative is ursodeoxycholic acid (UDCA)

[chemical structure]

In some embodiments the bile acid derivative is chenodeoxycholic acid (CDCA)

[chemical structure]

In some of the embodiments, the present disclosure pertains to the cocrystalline form of obeticholic acid and ursodeoxycholic acid, wherein the ratio of obeticholic acid to ursodeoxycholic acid is 2:1.

In some embodiments, the present disclosure pertains to the cocrystalline form of obeticholic acid and ursodeoxycholic acid, wherein the ratio of obeticholic acid to ursodeoxycholic acid is 1:1.

In some embodiments, the cocrystalline form of obeticholic acid and ursodeoxycholic acid can be characterized by a DSC having an endotherm onset at about 174° C.

In some embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at approximately 7.4, 13.8, 14.9, 16.7 and 17.8 degrees 2-theta (° 2θ) using Cu Kα radiation.

In one of the embodiments, the cocrystalline form of obeticholic acid and ursodeoxycholic acid, wherein the ratio of obeticholic acid to ursodeoxycholic acid is 2:1, is characterized by having X-ray powder diffraction (XRPD) comprising peaks at 7.4, 13.8, 14.9, 16.7 and 17.8±0.2° 2-theta (° 2θ) using Cu Kα radiation.

In some embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at approximately 7.4, 9.5, 13.8, 14.9, 15.2, 16.7, 17.7, 24.7 degrees 2-theta (° 2θ) using Cu Kα radiation.

In one of the embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at 7.4, 9.5, 13.8, 14.9, 15.2, 16.7, 17.7, 24.7±0.2° 2-theta (° 2θ) using Cu Kα radiation.

In one of the embodiments, the present disclosure, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at approximately 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 19.3, 19.8, 20.4, 20.7, 21.0, 22.3, 22.7, 23.0, 23.3, 24.3, 24.7 degrees 2-theta (° 2θ) using Cu Kα radiation.

In one of the embodiments, the present disclosure, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 19.3, 19.8, 20.4, 20.7, 21.0, 22.3, 22.7, 23.0, 23.3, 24.3, 24.7±0.2° 2-theta (° 2θ) using Cu Kα radiation.

In some embodiments, the cocrystalline form of obeticholic acid and ursodeoxycholic acid, wherein the ratio of obeticholic acid to ursodeoxycholic acid is 2:1, is also characterized by having a monoclinic crystal system with the following unit cell parameters: a=approximately 24.19 Å, b=approximately 11.88 Å, and c=approximately 25.59 Å.

In some embodiments, the present disclosure also relates to the cocrystalline forms of OCA which is characterized by stability on storage at 40° C./75% RH and 25° C./97% RH.

In some aspects, the present disclosure pertains to a pharmaceutical composition comprising the cocrystalline form of OCA and a co-former and a pharmaceutically acceptable diluent, excipient or carrier.

Some of the embodiments of the present disclosure pertains to a pharmaceutical composition comprising the cocrystalline form of OCA and a bile acid co-former and a pharmaceutically acceptable diluent, excipient or carrier.

In one of the embodiments, the present disclosure pertains to a pharmaceutical composition comprising the cocrystalline form of OCA and UDCA and a pharmaceutically acceptable diluent, excipient or carrier.

Some aspects of the present disclosure pertain to a method of treating or preventing an FXR-mediated disease or disorder in a subject in need thereof, comprising administering a therapeutically effective amount of the cocrystalline form of OCA and a co-former. In some embodiments the co-former is a bile acid. In one of the embodiments, the co-former is UDCA.

Some aspects of the present disclosure pertain to a method of modulating FXR activity in a subject in need thereof, comprising administering a therapeutically effective amount of the cocrystalline form of OCA and a co-former. In some embodiments the co-former is a bile acid. In one of the embodiments, the co-former is UDCA.

One of the aspects of the present disclosure relates to a process for preparing the cocrystalline form of OCA and a co-former, comprising:

(a) dissolving OCA and the co-former in a solvent to form a solution;

(b) optionally heating the resulted solution;

(c) cooling the solution and optionally applying the anti-solvent; and

(f) filtering the product from step (c) and drying the product under vacuum.

In one embodiment, the solvent is acetonitrile.

In another embodiment, the solvent is tetrahydrofuran and the anti-solvent is heptane.

Unless otherwise defined, all technical and scientific terms used herein have the same meaning as commonly understood by one of ordinary skill in the art to which this application belongs. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art. In the case of conflict, the present specification, including definitions, will control. In addition, the materials, methods, and examples are illustrative only and are not intended to be limiting.

Other features and advantages of the application will be apparent from the following detailed description and claims.

### BRIEF DESCRIPTION OF THE FIGURES

FIG. 1 shows XRPD diffractogram for obeticholic acid monoammonium salt Form 1.

FIG. 2 shows ¹H NMR spectrum for obeticholic acid monoammonium salt Form 1.

FIG. 3 shows DSC and TGA thermograms for obeticholic acid monoammonium salt Form 1.

FIG. 4 shows VT-XRPD analysis of obeticholic acid monoammonium salt Form 1

FIG. 5 shows XRPD stability for obeticholic acid monoammonium salt Form 1 post 7 days storage at elevated conditions (40° C./75% RH (relative humidity), and 40° C./96% RH)

FIG. 6 shows GVC kinetic plot for obeticholic acid monoammonium salt Form 1.

FIG. 7 shows GVC isotherm plot for obeticholic acid monoammonium salt Form 1.

FIG. 8 shows XRPD analysis post GVC experiment for obeticholic acid monoammonium salt Form 1.

FIG. 9 shows PLM micrograph of obeticholic acid monoammonium salt Form 1.

FIG. 10 shows XRPD diffractogram of OCA-UDCA cocrystal Form 1.

FIG. 11 shows ¹H NMR spectrum of OCA-UDCA cocrystal Form 1.

FIG. 12 shows DSC and TGA thermograms for OCA-UDCA cocrystal Form 1.

FIG. 13 shows GVC isotherm plot for OCA-UDCA cocrystal Form 1.

FIG. 14 shows GVC kinetic plot for OCA-UDCA cocrystal Form 1.

FIG. 15 shows XRPD analysis post GVC experiment for OCA-UDCA cocrystal Form 1.

FIG. 16 shows experimental and calculated XRPD traces of OCA-UDCA cocrystal Form 1.

FIG. 17 shows molecular configuration of OCA-UDCA (2:1) cocrystal Form 1.

FIG. 18 shows XRPD stability for UDCA cocrystal post 7 days storage at elevated conditions.

### DETAILED DESCRIPTION

The present disclose describes crystalline forms of obeticholic acid including crystalline salt and cocrystal forms suitable for further development.

As used in this disclosure and the accompanying claims, the indefinite articles “a” and “an” and the definite article “the” include plural as well as single referents, unless the context clearly indicates otherwise.

“Bile acid” as used herein are semi-synthetic steroid acids and streroid acids found predominantly in the bile of mammals and other vertebrates. Different molecular forms of bile acids can be synthesized in the liver by different species. Bile acids are conjugated with, for example, taurine or glycine in the liver, forming bile salts. Primary bile acids are those synthesized by the liver. Secondary bile acids result from bacterial actions in the colon.

Bile acids constitute a large family of molecules, composed of a steroid structure with four rings, a side-chain terminating in a carboxylic acid, and several hydroxyl groups, the number and orientation of which is different among the specific bile acids.

Obeticholic acid (OCA), is a semi-synthetic bile acid analogue which has the chemical structure 6α-ethyl-chenodeoxycholic acid.

[chemical structure]

Bile acids have four rings labeled A, B, C, and D, from the farthest to the closest to the side chain with the carboxyl group. The hydroxyl groups can be in either of two configurations: either up, termed beta (β; often drawn by convention as a solid line), or down, termed alpha (a; displayed as a dashed line).

[chemical structure]

Chenodeoxycholic acid (CDCA), also known as chenodesoxycholic acid, chenocholic acid and 3α,7α-dihydroxy-5β-cholan-24-oic acid, is a bile acid.

[chemical structure]

Ursodeoxycholic acid (UDCA), also known as ursodiol (USAN) is one of the secondary bile acids, which are metabolic byproducts of intestinal bacteria.

[chemical structure]

“Conjugate” as used herein are products of a conjugation reaction between bile acids and amino acids. Before the primary bile acids are secreted into the canalicular lumen they are conjugated via an amide bond at the terminal carboxyl group with either of the amino acids glycine or taurine. These conjugation reactions yield glycoconjugates and tauroconjugates, respectively. This conjugation process increases the amphipathic nature of the bile acids making them more easily secretable as well as less cytotoxic. The conjugated bile acids are the major solutes in human bile. For example, glyco- and tauroconjugates of CDCA can be represented by the following structures:

[chemical structure]

As used herein, the term “amino acid conjugates” refers to conjugates of a compound of the invention with any suitable amino acid. Taurine (—NH(CH2)2SO3H), glycine (—NHCH2CO2H), and sarcosine (—N(CH3)CH2CO2H) are examples of amino acid conjugates. Suitable amino acid conjugates of the compounds have the added advantage of enhanced integrity in bile or intestinal fluids. Suitable amino acids are not limited to taurine, glycine, and sarcosine.

As defined herein, the term “metabolite” refers to glucuronidated and sulphated derivatives of the compounds described herein, wherein one or more glucuronic acid or sulphate moieties are linked to compound of the invention. Glucuronic acid moieties may be linked to the compounds through glycosidic bonds with the hydroxyl groups of the compounds (e.g., 3-hydroxyl, 7-hydroxyl, 11-hydroxyl, and/or the hydroxyl of the R7 group).

Sulphated derivatives of the compounds may be formed through sulphation of the hydroxyl groups (e.g., 3-hydroxyl, 7-hydroxyl, 11-hydroxyl, and/or the hydroxyl of the R7 group). Examples of metabolites include, but are not limited to, 3-O-glucuronide, 7-O-glucuronide, 11-O-glucuronide, 3-O-7-O-diglucuronide, 3-O-11-O-triglucuronide, 7-O-11-O-triglucuronide, and 3-O-7-O-11-O-triglucuronide, of the compounds described herein, and 3-sulphate, 7-sulphate, 11-sulphate, 3,7-bisulphate, 3,11-bisulphate, 7,11-bisulphate, and 3,7,11-trisulphate, of the compounds described herein.

As used herein, the term “amorphous form” refers to a noncrystalline solid state form of a substance. Amorphous solids consist of disordered arrangements of molecules and do not possess a distinguishable crystal lattice.

The terms “crystalline form(s)” or “crystal form(s)” mean crystal structures in which a compound can crystallize. Different crystalline forms usually have different X-ray diffraction patterns, infrared spectra, melting points, density, crystal shape, optical and electrical properties, stability and solubility. Crystallization solvent, rate of crystallization, storage temperature, and other factors may cause one crystal form to dominate. Different crystalline forms or polymorphs may have different physical properties such as, for example, melting temperatures, heats of fusion, solubilities, dissolution rates and/or vibrational spectra as a result of the arrangement or conformation of the molecules in the crystal lattice.

The differences in physical properties exhibited by crystalline forms or polymorphs affect pharmaceutical parameters such as storage stability, compressibility and density (important in formulation and product manufacturing), and dissolution rates (an important factor in bioavailability). Differences in stability can also result from changes in chemical reactivity (e.g., differential oxidation, such that a dosage form discolors more rapidly when comprised of one polymorph or crystalline form than when comprised of another polymorph or crystalline form) or mechanical property (e.g., tablets crumble on storage as a kinetically favored crystalline from or polymorph converts to thermodynamically more stable crystalline form or polymorph) or both (e.g., tablets of one polymorph are more susceptible to breakdown at high humidity). Due to solubility/dissolution differences, in extreme cases, some crystalline or polymorphic transitions may result in lack of potency or, at the other extreme, toxicity. In addition, the physical properties of the crystal may be important in processing, for example, one crystalline form or polymorph might be more likely to form solvates or might be difficult to filter and wash free of impurities (e.g., particle shape and size distribution might be different between crystalline forms or polymorphs).

“Cocrystal(s)” as used herein is a crystalline material composed of two or more different molecules, typically drug and cocrystal former(s) (“coformer” or “coformers”), in the same crystal lattice that are associated by nonionic and noncovalent bonds.

Cocrystals are multi-component crystals based on hydrogen bonding interactions without the transfer of hydrogen ions to form salts. Bronsted acid-base chemistry is not a requirement for the formation of a cocrystal. Cocrystallization is a manifestation of directed self-assembly of different components. Cocrystals contain two or more components which are capable of forming a hydrogen bond. A general approach to coformer selection is by screening a predetermined library of pharmaceutically acceptable/approved compounds capable of forming a hydrogent bond with a particular API, e.g. OCA. The lead cocrystal candidate with superior physicochemical and pharmacological properties can then be developed into a dosage form.

In certain embodiments, the non-covalent forces are one or more hydrogen bonds (H-bonds). The coformer may be H-bonded directly to the API or may be H-bonded to an additional molecule which is bound to the API. The additional molecule may be H-bonded to the API or bound ionically or covalently to the API. The additional molecule could also be a different API. In certain embodiments, the cocrystals may include one or more solvate molecules in the crystalline lattice, i.e., solvates of cocrystals, or a cocrystal further comprising a solvent or compound that is a liquid at room temperature. In certain embodiments, the cocrystals may be a cocrystal between a coformer and a salt of an API. In certain embodiments, the non-covalent forces are pi-stacking, guest-host complexation and/or van der Waals interactions. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads.

In certain embodiments, the cocrystals include an acid addition salt or base addition salt of an API.

Cocrystals are readily distinguished from salts because unlike salts, their components are in a neutral state and interact nonionically. In addition, cocrystals differ from polymorphs, which are defined as including only single-component crystalline forms that have different arrangements or conformations of the molecules in the crystal lattice, amorphous forms, and multicomponent phases such as solvate and hydrate forms. Instead, cocrystals are more similar to solvates, in that both contain more than one component in the lattice. From a physical chemistry perspective, cocrystals can be viewed as a special case of solvates and hydrates, wherein the second component, the coformer, is nonvolatile. Therefore, cocrystals can be classified as a special case of solvates in which the second component is nonvolatile.

Cocrystals may include one or more solvent/water molecules in the crystal lattice. Co-crystals often rely on hydrogen-bonded assemblies between neutral molecules of API and other component. For nonionizable compounds cocrystals enhance pharmaceutical properties by modification of chemical stability, moisture uptake, mechanical behaviour, solubility, dissolution rate and bioavailability.

Cocrystals can be tailored to enhance drug product bioavailability and stability and to enhance the processability of APIs (active pharmaceutical ingredients) during drug product manufacture. Another advantage of cocrystals is that they generate a diverse array of solid-state forms for APIs that lack ionizable functional groups, which is a prerequisite for salt formation. Cocrystals are crystalline materials composed of two or more different molecules, typically drug and cocrystal formers (“coformers”), in the same crystal lattice. Pharmaceutical cocrystals have opened up opportunities for engineering solid-state forms beyond conventional solid-state forms of an active pharmaceutical ingredient (API), such as salts and polymorphs. Cocrystals can be tailored to enhance drug product bioavailability and stability and to enhance the processability of APIs during drug product manufacture. Another advantage of cocrystals is that they generate a diverse array of solid-state forms for APIs that lack ionizable functional groups, which is a prerequisite for salt formation.

A cocrystal with a pharmaceutically acceptable coformer can be a pharmaceutical cocrystal and has a regulatory classification similar to that of a polymorph of the API. Drug products that are designed to contain a new cocrystal are considered analogous to a new polymorph of the API. A cocrystal that is composed of two or more APIs (with or without additional inactive coformers) will be treated as a fixed-dose combination product (Regulatory Classification of Pharmaceutical Co-Crystals; Guidance for Industry; U.S. Department of Health and Human Services Food and Drug Administration Center for Drug Evaluation and Research (CDER); Pharmaceutical Quality/CMC Revision 1; August 2016).

Pharmaceutical cocrystals have gained recent prominence in research reports demonstrating that complexation of an active pharmaceutical ingredient (API) with another molecule can produce a solid form with improved properties, such as aqueous solubility, dissolution, hygroscopicity, bioavailability, stability, increase of melting point, purity of API, and developability. Pharmaceutical cocrystals are cocrystals of a therapeutic compound, e.g., an active pharmaceutical ingredient (API), and one or more non-volatile compound(s) (referred to herein as coformer).

“Coformer(s)” or “cocrystal former(s)” or “cocrystallization partner(s)” as used herein is a pharmaceutically-acceptable molecule capable of forming an H-bond with an API. The H-bond is formed between the H-bond donor and the H-bond acceptor. Cocrystal former (CCF) is specifically selected to impart certain advantageous attributes to the API.

The coformer selection which is compatible with API is one of the challenges in cocrystal development. A general approach to coformer selection is by “tactless” cocrystal screening, whereby a predetermined library of pharmaceutically acceptable/approved compounds is used to attempt cocrystallization. In cocrystal development one of the approach of coformer selection is based on trial and error. Other approaches can be supramolecular synthon approach which utilizes Cambridge Structural Database (CSD) to effectively prioritize coformers for crystal form screening, Hansen solubility parameter and knowledge of hydrogen bonding between coformer and API.

A coformer in a pharmaceutical cocrystal is typically selected from non-toxic pharmaceutically acceptable molecules, such as, for example, food additives, preservatives, pharmaceutical excipients, or other APIs.

The ratio of API to coformer may be stoichiometric or non-stoichiometric. In one embodiment, the ratio of API to coformer is about 5:4, 5:3, 5:2, 5:1, 4:5, 4:3, 4:1, 3:5, 3:4, 3:2, 3:1, 2:5, 2:3, 2:1, 1:1.

In one embodiment, the cocrystal comprises more than one coformers. In one embodiment, the cocrystal comprises two coformers.

“Salts” as used herein are any of numerous compounds that result from replacement of part or all of the acid hydrogen of an acid by a metal or a radical acting like a metal: an ionic or electrovalent crystalline compound. For ionizable compounds, preparation of salt forms using pharmaceutically acceptable acids and bases is a common strategy to improve bioavailability. Like the parent compound, pharmaceutical salts may exist in several polymorphic, solvated and/or hydrated forms. The selection of an appropriate salt form for a potential drug candidate is an opportunity to modulate its characteristics to improve bioavailability, stability, manufacturability, and patient compliance. Base addition salts include, but are not limited to, inorganic bases such as sodium, potassium, lithium, ammonium, calcium and magnesium salts, and organic bases such as primary, secondary and tertiary amines (e.g., isopropylamine, trimethyl amine, diethyl amine, tri (iso-propyl) amine, tri(n-propyl) amine, ethanolamine, 2-dimethylaminoethanol, tromethamine, lysine, arginine, histidine, procaine, hydrabamine, choline, betaine, ethylenediamine, glucosamine, N-alkylglucamines, theobromine, purines, piperazine, piperidine, morpholine, and N-ethylpiperidine).

“Counter ion” as used herein is an ion that accompanies an ionic species in order to maintain electric neutrality. In case of OCA, counterions may include, for example, sodium, potassium, magnesium, ethanolamine, and ammonium ions.

Pharmaceutically acceptable salts of bile acids include, but are not limited to, the alkali metal salts, alkaline earth metal salts, ammonium salts, alkylammonium salts containing, for example, 1-6 carbon atoms or dialkylammonium salts containing 1-6 carbon atoms in each alkyl group, trialkylammonium salts containing 1-6 carbon atoms in each alkyl group and tetraalkylammonium salts containing 1-6 carbon atoms in each alkyl group. Alkali metal salts include sodium and potassium salts. Alkaline earth metal salts include calcium and magnesium salts. Suitable alkyl groups include methyl, ethyl, propyl, butyl, pentyl and hexyl. Where more than one alkyl group is present the groups may be the same or different.

“Polymorphs” or “polymorphic forms” are different crystalline forms of the same active pharmaceutical ingredient (API). This may include solvation or hydration products (also known as pseudopolymorphs) and amorphous forms.

Polymorphism is often characterized as the ability of a drug substance to exist as two or more crystalline phases that have different arrangements and/or conformations of the molecules in the crystal lattice. Polymorphism refers to the occurrence of different crystalline forms of the same drug substance. Polymorphism in this commentary is defined as in the International Conference on Harmonization (ICH) Guideline Q6A, to include solvation products and amorphous forms.

As used herein, the term “solvate” means solvent addition form or forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrates. The compounds of the present application may exist in either hydrated or unhydrated (the anhydrous) form or as solvate with other solvent molecule(s) or in an unsolvated form.

Nonlimiting examples of hydrates include monohydrates, dihydrates, etc. Nonlimiting examples of solvates include DCM (dichloromethane) solvates, MEK (methylethyl ketone) solvates, THF (tetrahydrofuran) solvates, etc. If the solvent is water, the solvate formed is a hydrate, and when the solvent is alcohol, the solvate formed is an alcoholate. Hydrates are formed by the combination of one or more molecules of water with one of the substances in which the water retains its molecular state as H2O, such combination being able to form one or more hydrate.

Solvates are crystalline solid adducts containing either stoichiometric or nonstoichiometric amounts of a solvent incorporated within the crystal structure. If the incorporated solvent is water, the solvates are also commonly known as hydrates.

Polymorphs and/or solvates of a pharmaceutical solid can have different chemical and physical properties such as melting point, chemical reactivity, apparent solubility, dissolution rate, optical and electrical properties, vapor pressure, and density. These properties can a direct impact on the process-ability of drug substances and the quality/performance of drug products, such as stability, dissolution, and bioavailability. A metastable pharmaceutical solid form can change crystalline structure or solvate/desolvate in response to changes in environmental conditions, processing, or over time.

Since polymorphs exhibit certain differences in physical (e.g., powder flow and compactability, apparent solubility and dissolution rate) and solid state chemistry (reactivity) attributes that relate to stability and bioavailability it is essential that the product development.

The compounds provide herein may also contain an unnatural proportion of an atomic isotope at one or more of the atoms that constitute such a compound. For example, the compound may be radiolabeled with radioactive isotopes, such as for example deuterium (²H), tritium (³H) or carbon-14 (¹⁴C). Radiolabeled compounds are useful as therapeutic agents, e.g., cancer therapeutic agents, research reagents, e.g., binding assay reagents, and diagnostic agents, e.g., in vivo imaging agents. All isotopic variations of the compounds provided herein, whether radioactive or not, are intended to be encompassed herein. In certain embodiments, a compound provided herein contains unnatural proportion(s) of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N) nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), and chlorine-37 (³⁷Cl). In certain embodiments, a compound provided herein contains unnatural proportion(s) of one or more isotopes in a stable form, that is, non-radioactive. In certain embodiments, a compound provided herein contains unnatural proportion(s) of one or more isotopes in an unstable form, that is, radioactive. In certain embodiments, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill in the art. In certain embodiments, a compound provided herein contains unnatural proportions of deuterium (D).

As used herein, a compound is “stable” where significant amounts of degradation products are not observed under constant conditions of humidity (e.g., about 10%, about 20%, about 30%, about 40%, about 50%, about 60%, about 70%, about 75%, about 80%, about 85%, about 90%, and about 95% RH), light exposure, and/or temperatures (e.g., higher than about 0° C., e.g., about 20° C., about 25° C., about 30° C., about 35° C., about 40° C., about 45° C., about 50° C., about 55° C., about 60° C., about 65° C., and about 70° C.) over a certain period (e.g., one week, two weeks, three weeks, and four weeks). A compound is not considered to be stable at a certain condition when degradation impurities appear or an area percentage (e.g., AUC as characterized by HPLC) of existing impurities begins to grow. The amount of degradation growth as a function of time is important in determining compound stability.

As used herein, the term “mixing” means combining, blending, stirring, shaking, swirling, or agitating. The term “stirring” as used herein can mean mixing, shaking, agitating, or swirling. The term “agitating” as used herein can mean mixing, shaking, stirring, or swirling.

Techniques for characterizing crystalline forms or polymorphs include, but are not limited to, differential scanning calorimetry (DSC), X-ray powder diffractometry (XRPD), single crystal X-ray diffractometry, vibrational spectroscopy (e.g., IR and Raman spectroscopy), TGA (Thermogravimetric analysis), DTA (Differential thermal analysis), DVS (Dynamic vapour sorption), solid state NMR, hot stage optical microscopy, scanning electron microscopy (SEM), electron crystallography and quantitative analysis, particle size analysis (PSA), surface area analysis, solubility studies, and dissolution studies.

Unless explicitly indicated otherwise, the terms “approximately” and “about” are synonymous. In one embodiment, “approximately” and “about” refer to recited amount, value, or duration, e.g, ±20%, ±15%, ±10%, ±8%, ±6%, ±5%, ±4%, ±2%, ±1%, or ±0.5% of that value. In another embodiment, “approximately” and “about” refer to listed amount, value, or duration ±10%, ±8%, ±6%, ±5%, ±4%, or ±2%. In yet another embodiment, “approximately” and “about” refer to listed amount, value, or duration ±5%. In yet another embodiment, “approximately” and “about” refer to listed amount, value, or duration ±2%.

When the terms “approximately” and “about” are used when reciting XRPD peaks, these terms refer to the recited X-ray powder diffraction peak ±0.3° 2θ (theta), ±0.2° 20 (theta), or ±0.1° 2θ (theta). In another embodiment, the terms “approximately” and “about” refer to the listed X-ray powder diffraction peak ±0.2° 2θ (theta). In another embodiment, the terms “approximately” and “about” refer to the listed X-ray powder diffraction peak ±0.1° 2θ (theta).

When the terms “approximately” and “about” are used when reciting temperature or temperature range, these terms refer to the recited temperature or temperature range ±5° C., 2° C., or ±1° C. In another embodiment, the terms “approximately” and “about” refer to the recited temperature or temperature range ±2° C. In another embodiment, the terms “approximately” and “about” refer to the recited temperature or temperature range ±1° C.

As used herein, “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained.

A “pharmaceutical composition” is a formulation containing an active agent in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient in a unit dose of composition is an effective amount and varies according to the particular treatment involved.

“Pharmaceutically acceptable diluent/excipient/carrier” means a diluent/excipient/carrier that is useful in preparing a pharmaceutical composition that is generally safe, non-toxic, and neither biologically nor otherwise undesirable, and is acceptable for veterinary use as well as human pharmaceutical use. A “pharmaceutically acceptable diluent/excipient/carrier” as used in the specification and claims includes both one and more than one such diluent/excipient/carrier.

Pharmaceutically acceptable carriers, for example, or excipients have met the required standards of toxicological and manufacturing testing and/or are included on the Inactive Ingredient Guide prepared by the U.S. Food and Drug administration. The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier is “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “treating” or “treat” as used herein refers to any indicia of success in the treatment or amelioration of a disease or disorder. Treating can include, for example, ameliorating, i.e., causing regression of the disease state or condition, relieving, lessening, reducing, eliminating, modulating, or alleviating the severity of one or more symptoms of a disease or disorder, or it can include reducing the frequency with which symptoms of a disease or disorder are experienced by a patient. “Treating” can also refer to reducing or eliminating a condition of a part of the body, such as a cell, tissue or bodily fluid (e.g., blood).

As used herein, the term “prevent” or “preventing” refers to the partial or complete prevention of a disease or disorder in an individual or in a population, or in a part of the body, such as a cell, tissue or bodily fluid (e.g., blood). The term “prevention” does not establish a requirement for complete prevention of a disease or disorder in the entirety of the treated population of individuals or cells, tissues or fluids of individuals. The term “preventing”, as used herein, also refers to completely or almost completely stop a disease state or condition, from occurring in a patient or subject, especially when the patient or subject is predisposed to such or at risk of contracting a disease state or condition. Preventing can also include inhibiting, i.e., arresting the development, of a disease state or condition, and relieving or ameliorating, i.e., causing regression of the disease state or condition, for example when the disease state or condition may already be present.

The term “prophylactically effective amount” means an amount (quantity or concentration) of a compound of the present invention, or a combination of compounds, that is administered to prevent or reduce the risk of a disease—in other words, an amount needed to provide a preventative or prophylactic effect. The amount of the present compound to be administered to a subject will depend on the particular disorder, the mode of administration, co-administered compounds, if any, and the characteristics of the subject, such as general health, other diseases, age, sex, genotype, body weight, and tolerance to drugs. The skilled artisan will be able to determine appropriate dosages depending on these and other factors.

The term “therapeutically effective amount” or “effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. A disease or disorder to be treated or prevented can be, for example, a liver disease or disorder.

The phrase “reducing the risk of”, as used herein, refers to lowering the likelihood or probability of a central nervous system disease, inflammatory disease and/or metabolic disease from occurring in a patient, especially when the subject is predisposed to such occurrence.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary depending upon various factors, including but not limited to the dosage form employed, sensitivity of the patient, and the route of administration.

“Combination therapy” (or “co-therapy”) refers to the administration of a compound of the invention and at least a second agent as part of a specific treatment regimen intended to provide the beneficial effect from the co-action of these therapeutic agents (i.e., the compound of the invention and at least a second agent). The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present application. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also embraces the administration of the therapeutic agents as described above in further combination with other biologically active ingredients and non-drug therapies (e.g., surgery or mechanical treatments). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Crystalline Forms of Disclosure

Obeticholic acid (OCA) can form crystalline or cocrystalline forms, such as salts and cocrystals with relevant partner molecules.

Obeticholic acid and its solid forms can be prepared according to known methods described, for example, in U.S. Pat. Nos. 7,994,352 and 9,238,673, the entireties of which are incorporated herein by reference.

In one of the embodiments, the present disclosure pertains to cocrystalline forms of obeticholic acid (OCA) and a pharmaceutically acceptable co-former

[chemical structure]

Coformer that can be considered for cocrystallization with OCA include, but are not limited to oxalic acid, maleic acid, glutamic acid, pamoic acid, malonic acid, 2,5-dihydroxybenzoic acid, L-tartaric acid, fumaric acid, DL-mandelic acid, ascorbic acid, benzoic acid, succinic acid, trans-cinnamic acid, adipic acid, nicotinic acid, stearic acid, sorbic acid,

Saccharine, urea, 3-hydroxybenzoic acid, glycine, cholic acid, deoxycholic acid, ursodeoxycholic acid, chenodeoxycholic acid, and other bile acids and their derivatives as, for example, described in U.S. Pat. Nos. 7,812,011, 7,932,244, and 8,445,472 and U.S. Patent Application Publication No. 2014/0371190, and other pharmaceutically acceptable and compatible compounds.

In some embodiments, the present disclosure pertains to the cocrystalline forms of OCA, wherein the co-former is a bile acid derivative.

Bile acids can be used as coformers in cocrystallization with OCA. Such bile acids comprise cholic acid, deoxycholic acid, ursodeoxycholic acid, chenodeoxycholic acid, and other semi-synthetic bile acids and their derivatives as, for example, described in U.S. Pat. Nos. 7,812,011, 7,932,244, and 8,445,472 and U.S. Patent Application Publication No. 2014/0371190.

In some embodiments the bile acid derivative is chenodeoxycholic acid (CDCA)

[chemical structure]

In some embodiments, the bile acid derivative is ursodeoxycholic acid (UDCA)

[chemical structure]

The ratio of obeticholic acid to ursodeoxycholic acid in the cocrystal can be 1:2 or 1:1.

In some of the embodiments, the present disclosure pertains to the cocrystalline form of obeticholic acid and ursodeoxycholic acid, wherein the ratio of obeticholic acid to ursodeoxycholic acid is 2:1.

In some embodiments, the present disclosure pertains to the cocrystalline form of obeticholic acid and ursodeoxycholic acid, wherein the ratio of obeticholic acid to ursodeoxycholic acid is 1:1.

In some embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at approximately 7.4, 13.8, 14.9, 16.7 and 17.8 degrees 2-theta (° 2θ) using Cu Kα radiation.

In some embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at 7.4, 13.8, 14.9, 16.7 and 17.8±0.2° 2-theta (° 2θ) using Cu Kα radiation.

In some embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at approximately 7.4, 9.5, 13.8, 14.9, 15.2, 16.7, 17.7, 24.7 degrees 2-theta (° 2θ) using Cu Kα radiation.

In one of the embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at 7.4, 9.5, 13.8, 14.9, 15.2, 16.7, 17.7, 24.7±0.2° 2-theta (° 2θ) using Cu Kα radiation.

In some embodiments, the cocrystalline form of obeticholic acid and ursodeoxycholic acid, wherein the ratio of obeticholic acid to ursodeoxycholic acid is 2:1, is characterized by having X-ray powder diffraction (XRPD) comprising peaks at approximately 3.6, 7.4, 8.3, 8.7, 9.5, 10.3, 10.9, 11.2, 11.9, 12.8, 13.8, 14.9, 15.2, 16.7, 17.7, and 24.7 degrees 2-theta using Cu Kα radiation.

In one of the embodiments, the cocrystalline form of obeticholic acid and ursodeoxycholic acid, wherein the ratio of obeticholic acid to ursodeoxycholic acid is 2:1, is characterized by having X-ray powder diffraction (XRPD) comprising peaks at 3.6, 7.4, 8.3, 8.7, 9.5, 10.3, 10.9, 11.2, 11.9, 12.8, 13.8, 14.9, 15.2, 16.7, 17.7, and 24.7±0.2° 2-theta using Cu Kα radiation.

In some embodiments, the cocrystalline form of obeticholic acid and ursodeoxycholic acid, wherein the ratio of obeticholic acid to ursodeoxycholic acid is 2:1, is characterized by having X-ray powder diffraction (XRPD) comprising peaks at approximately 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 23.0, 23.3, 24.3, and 24.7 degrees 2-theta using Cu Kα radiation.

In one of the embodiments, the cocrystalline form of obeticholic acid and ursodeoxycholic acid, wherein the ratio of obeticholic acid to ursodeoxycholic acid is 2:1, is characterized by having X-ray powder diffraction (XRPD) comprising peaks at 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 23.0, 23.3, 24.3, and 24.7±0.2° 2-theta using Cu Kα radiation.

In one of the embodiments, the cocrystalline form of obeticholic acid and ursodeoxycholic acid, wherein the ratio of obeticholic acid to ursodeoxycholic acid is 2:1 may be characterized by having X-ray powder diffraction (XRPD) comprising peaks at approximately 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 19.3, 19.8, 20.4, 20.7, 21.0, 22.3, 22.7, 23.0, 23.3, 24.3, 24.7 degrees 2-theta (° 2θ) using Cu Kα radiation.

In one of the embodiments, the cocrystalline form of obeticholic acid and ursodeoxycholic acid, wherein the ratio of obeticholic acid to ursodeoxycholic acid is 2:1 is characterized by having X-ray powder diffraction (XRPD) comprising peaks at 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 19.3, 19.8, 20.4, 20.7, 21.0, 22.3, 22.7, 23.0, 23.3, 24.3, 24.7±0.2° 2-theta using Cu Kα radiation.

In some of the embodiments, the cocrystalline form of obeticholic acid and ursodeoxycholic acid is characterized by having X-ray powder diffraction as shown in FIG. 10.

In certain embodiments, the cocrystalline form of obeticholic acid and ursodeoxycholic acid, wherein the ratio of obeticholic acid to ursodeoxycholic acid is 2:1, is characterized by having a monoclinic crystal system with the following unit cell parameters: a=approximately 24.19 Å, b=approximately 11.88 Å, and c=approximately 25.59 Å.

In some embodiments, the cocrystalline form of obeticholic acid and ursodeoxycholic acid is characterized by a DSC thermogram having an endotherm onset occurring at about 174° C. The melting process of cocrystalline form of obeticholic acid and ursodeoxycholic acid is characterized by having a DSC thermogram as shown in FIG. 12.

### Preparation of Crystalline Forms

Solid forms provided herein can be prepared by the methods described herein, or by techniques, including, but not limited to, heating, cooling, freeze drying, spray drying, lyophilization, quench cooling the melt, rapid solvent evaporation, slow solvent evaporation, solvent recrystallization, antisolvent addition, slurry recrystallization, crystallization from the melt, desolvation, recrystallization in confined spaces, such as, e.g., in nanopores or capillaries, recrystallization on surfaces or templates, such as, e.g., on polymers, recrystallization in the presence of additives, such as, e.g., cocrystal counter-molecules, desolvation, dehydration, rapid cooling, slow cooling, exposure to solvent and/or water, drying, including, e.g., vacuum drying, vapor diffusion, sublimation, grinding (including, e.g., cryo-grinding and solvent-drop grinding), microwave-induced precipitation, sonication-induced precipitation, laser-induced precipitation, and precipitation from a supercritical fluid. The particle size of the resulting solid forms, which can vary (e.g., from nanometer dimensions to millimeter dimensions), can be controlled, e.g., by varying crystallization conditions, such as, e.g., the rate of crystallization and/or the crystallization solvent system, or by particle-size reduction techniques, e.g., grinding, milling, micronizing, or sonication.

In one of the aspects, the present disclosure relates to a process for preparing a crystalline form of OCA comprising:

(a) dissolving OCA and the counterion or co-former in a solvent to form a solution;

(b) optionally heating the resulted solution;

(c) cooling the solution and optionally applying the anti-solvent; and

(f) filtering the product from step (c) and drying the product (e.g., crystalline form) under vacuum.

In certain embodiments, crystalline forms (e.g., cocryslats) can be prepared using solid-state methods such as solid-state grinding and solvent-drop grinding. In certain embodiments, crystalline forms (e.g., cocrystals) can be prepared using high-throughput screening. In certain embodiments, crystalline forms (e.g., cocrystals) can be prepared using solution-based crystallization.

In certain embodiments, slurry crystallization is effected by adding solvent or solvent mixtures to a solid substrate, and the slurry is stirred, and optionally heated to various temperatures. In certain embodiments, the slurry is heated at about 25° C., about 50° C., about 80° C., or about 100° C. In certain embodiments, upon heating and cooling, the residual solvents of the slurry can be removed by wicking, or other suitable methods, such as filtration, centrifugation, or decantation, and the crystals can be dried in air or under vacuum.

In certain embodiments, evaporation crystallization of solid forms of OCA is effected by adding a solvent or solvent mixture to a solid substrate, and allowing the solvent or solvent mixture to evaporate under ambient conditions. In certain embodiments, the residual solvent can be removed by wicking, or other suitable methods, such as filtration, centrifugation, or decantation, and the crystals can be dried in air or under vacuum.

In certain embodiments, precipitation crystallization is effected by adding a solvent or solvent mixture to a solid substrate, and subsequently adding an anti-solvent. In certain embodiments, the resultant mixture stands for a period of time, e.g., overnight, and under certain conditions, for example at room temperature. In certain embodiments, the residual solvent can be removed by wicking, or other suitable methods, such as filtration, centrifugation, or decantation, and the crystals can be dried in air or under vacuum.

In some embodiments, crystallization can be effected by Common Class 3 solvents including, but not limited to acetic acid, acetone, 1- and 2-butanol, butyl acetate, dimethyl sulfoxide (DMSO), ethanol, ethyl acetate, ethyl ether, ethyl formate, heptane, isobutyl acetate, methyl acetate, methyl ethyl ketone (MEK), methyl isobutyl ketone (MIBK), methyl t-butyl ether, pentane, 1- and 2-propanol, and 1- and 2-propyl acetate.

In some embodiments, crystallization can be effected by Common Class 2 solvents including, but not limited to acetonitrile, chloroform, cyclohexane, dichloromethane (DCM), 1,2-dichloroethane, 1,2-dimethoxyethane, dimethylacetamide, N,N-dimethylformamide (DMF), 1,4-dioxane, 2-ethoxyethanol, 2-methoxyethanol, hexane, methanol, methyl butyl ketone, methylcyclohexane, nitromethane, sulfolane, tetrahydrofuran (THF), tetralin, toluene, and xylene.

In some embodiments, crystallization can be effected by a solvent system comprising one or more of a Common Class 2 solvent and one or more of a Commen Class 3 solvent. In some embodiments, suitable solvents or solvent systems comprise one or more of the following solvents: acetone, N,N-dimethylformamide (DMF), dimethyl sulfoxide (DMSO), tetrahydrofuran (THF), water, methanol, ethanol, isopropanol, or acetonitrile.

In certain embodiments, cooling crystallization is effected by adding a solvent or solvent mixture to a solid substrate at elevated temperature, and allowing the resultant mixture to stand for a period of time at a reduced temperature. In certain embodiments, the elevated temperature is, for example, about 30° C., about 40° C., about 50° C., about 60° C., about 70° C., or about 80° C. In certain embodiments, the reduced temperature is, for example, about 15° C., about 10° C., about 5° C., about 0° C., about −5° C., about −10° C., about −15° C., or about −20° C. The residual solvent can be removed by wicking, or other suitable methods, such as filtration, centrifugation, or decantation, and the crystals can be dried in air or under vacuum.

In certain embodiments, the cooling rate is, for example, between about 5° C./min and about 0.05° C./min. Specifically, the cooling rate can be about 5° C./min, about 4° C./min, about 3° C./min, about 2° C./min, about 1° C./min, about 0.9° C./min, about 0.8° C./min, about 0.7° C./min, about 0.6° C./min about 0.5° C./min. about 0.4° C./min, about 0.3° C./min, about 0.2° C./min, about 0.1° C./min or about 0.05° C./min.

### Preparation of Crystalline Salts

In one of the aspects, the present disclosure relates to a process for preparing crystalline salts of OCA comprising:

(a) dissolving OCA and the counterion in a solvent to form a solution;

(b) optionally heating the resulted solution;

(c) cooling the solution and optionally applying the anti-solvent; and

(f) filtering the product from step (c) and drying the product (e.g., crystalline salt) under vacuum.

In some embodiments, crystalline salt screening or preparation procedures include cooling with Common Class 2 solvents (e.g., acetonitrile and isopropyl alcohol or 2-propanol (IPA)). In some embodiments, OCA is dissolved in Class 2 solvent (e.g., acetonitrile) at about 40-60° C. (e.g., 50° C.) and about 1.1 eq of counter-ion solution is added. After precipitation, the samples are cooled down to about 0-10° C. (e.g., 5° C.) at about 1° C./min and stirred at this temperature for about one to three hours (e.g., one hour). Some experiments are performed with a slower cooling rate of about 0.1° C./min from about 50° C. to about 5° C. Solids are filtered, dried under vacuum and analysed (e.g., by XRPD).

In some embodiments, the cooling procedure for salt screening in Class 2 solvent (e.g., isopropyl alcohol) is performed with about 0.1-0.5 mL (e.g. 0.15 mL, 0.2 mL, 0.25 mL or 0.3 mL) of solvent. In some embodiments, solutions are divided into two or more portions for slow evaporation and/or anti-solvent addition experiments as discussed herein.

In some embodiments maturation is carried out at about 20-30° C. (e.g., about 25° C.) or about 40-60° C. (e.g., about 50° C.). The amorphous samples from cooling experiments in Class 2 solvent (e.g., acetonitrile) are re-suspended in about 200-400 μL of solvent (e.g., about 300 μL of acetonitrile). Samples are typically stirred at about 500 rpm at about 25 to about 50° C. (about 8 h cycle) for about 10-30 hours (e.g., about 20 hours). Suspensions are dried for about 5 hours under vacuum (at about 25° C.) and analysed by XRPD. The solution obtained can be split into two or more portions for slow evaporation and/or anti-solvent addition experiments as discussed herein.

In some embodiments maturation is performed at about 50° C. In some experiments, OCA is dissolved in Class 2 solvent (e.g., acetonitrile) at about 50° C. and about 1.1 eq of counter-ion solution is added. Precipitation can occur after the counterion is added. The samples are typically left stirring at about 50° C. and about 250 rpm for about 24 hours. Suspensions are filtered, dried under vacuum for about 5 hours under vacuum (e.g., at about 25° C.) and analysed (e.g., by XRPD).

In certain embodiments, crystallization of salts of OCA is effected by cooling salt samples in a solvent (e.g., acetonitrile and isopropyl alcohol (IPA)) to about 10-0° C. (e.g., about 5° C.) at about 3-0.5° C./min (e.g., 1° C./min) under stirring for about 0.5-3 hours (e.g., one hour) or with a slower cooling rate of about 0.4-0.05° C./min (e.g., 0.1° C./min) from about 20-70° C. (e.g., about 50° C.) to about 10-0° C. (e.g., about 5° C.). Salt samples are typically prepared by dissolving obeticholic acid in a solvent at about 20-70° C. (e.g., about 50° C.) and adding approximately equal molar amount (e.g., about 1.1 eq) of counter-ion to the solution. Cooling is initiated upon formation of the precipitate. Crystalline material is filtered, dried under vacuum and analysed (e.g., XRPD).

Suitable counterions include, but are not limited to sodium, potassium, calcium, magnesium, L-arginine, choline, L-lysine, ethanolamine, ammonia, N-ethylglucamine, and N-methylglucamine.

In certain embodiments, crystallization of salts of OCA is effected by slow evaporation of the solvent (e.g., acetonitrile or IPA). In certain embodiments, the slow evaporation is effected by inserting a microneedle through the lid of a sample vial or container. Solutions are typically obtained after maturation at about 25-50° C. Slow solvent evaporation time vary and can be up to about one to five weeks. In some embodiments, sample solutions (e.g., about 50 μL) from cooling experiments can be placed in sealed vials with a microneedle inserted through the lid to allow slow evaporation of solvent. Solutions obtained after maturation at about 25° C.-50° C. (e.g., in acetonitrile) (e.g., about 500 μL) can be also subjected to slow evaporation for over a week or more (e.g., two, three, four, or five weeks). After slow evaporation, samples can be analyzed (e.g., by XRPD). In certain embodiments, crystallization of salts of OCA is effected by addition of an antisolvent. The salt solutions can be held at about 25-50° C. and then treated with an anti-solvent, e.g. water. The samples are then cooled to about 5° C. at about 1° C./min under stirring at about 300-600 rpm (e.g., at about 500 rpm). After cooling, samples are allowed to evaporate to dryness under ambient conditions and analyzed (e.g., by XRPD).

In some embodiments, the OCA solutions (e.g. after an aliquot is taken for slow evaporation experiment) are held at about 50° C. for about 15 min and then treated with an anti-solvent (e.g. water). The samples are cooled to about 5° C. at about 1° C./min while stirring at about 500 rpm. After cooling, samples are left to evaporate to dryness under ambient conditions. Powdered samples are further analyzed (e.g., by XRPD).

In some embodiments, suitable antisolvents can be non-polar solvents, which include, but are not limited to cyclohexane, heptane, hexane, methylcyclohexane, octane (or isooctane), pentane, tetralin, toluene, and xylene.

In some embodiments, obeticholic acid salts can be prepared by dissolving OCA in an aqueous counter-ion equal molar amount (e.g., about 1.1 eq) solution, heating the resultant mixture at about 30-70° C. (e.g., 50° C.) for about 10-90 minutes (e.g. 30 minutes) and cooled to about 10-0° C. (e.g., 5° C.) at about 1-0.05° C./min (e.g., 0.1° C./min) under stirring with a stirring speed of about 200-600 rpm (e.g., 300 rpm). In certain embodiments, when samples remain solutions, they can be lyophilised. The lyophilised solids are then suspended in a solvent (e.g., heptane) and stirred at about 20-60° C. (e.g., 50° C.) at about 200-600 rpm (e.g., 300 rpm) for up to 10 days (e.g. about 5 days). In one of the embodiments of the present disclosure, the crystalline forms (e.g. salts) are obtained from a water/heptane solvent system. Obeticholic acid is dissolved in an aqueous counter-ion solution. The samples are heated at about 50° C. for about 30 minutes and cooled to about 5° C. at about 0.1° C./minute. Stirring speed of about 300 rpm is maintained throughout the experiments. The samples that remained solutions can be topped up with water (about 0.5 mL) and lyophilised. The lyophilised solids are suspended in heptane (e.g., about 0.6 mL) and left stirring at about 50° C. at about 300 rpm for 1-10 days (e.g., about 2, 3, 4, or 5 days). Solids can be filtered under partial vacuum (suction) filtration and analysed (e.g., by XRPD).

Crystalline forms (e.g., salts or cocrystals) of the present disclosure can be characterized by, for example, differential scanning calorimetry (DSC), X-ray powder diffractometry (XRPD), single crystal X-ray diffractometry, vibrational spectroscopy (e.g., IR and Raman spectroscopy), TGA (Thermogravimetric analysis), DTA (Differential thermal analysis), GVS (Gravimetric vapor sorption), solid state NMR, hot stage optical microscopy, scanning electron microscopy (SEM), electron crystallography and quantitative analysis, particle size analysis (PSA), surface area analysis, solubility studies, and dissolution studies.

### Preparation of Cocrystals

One of the aspects of the present disclosure relates to a process for preparing the cocrystalline form of OCA and a co-former, comprising:

(a) dissolving OCA and the co-former in a solvent to form a solution;

(b) optionally heating the resulted solution;

(c) cooling the solution and optionally applying the anti-solvent; and

(f) filtering the product from step (c) and drying the product under vacuum.

In certain embodiments, preparation of cocrystals of OCA is effected solvent drop grinding method. In some embodiments, a mixture of obeticholic acid and a coformer in equal molar amount (e.g., about 1.1 eq) in a stainless steel grinding jar equipped with a grinding ball (e.g., one 7 mm grinding ball) is wetted with a solvent (e.g., acetonitrile, nitromethane, or heptane) and ground for about 0.5-5 hours (e.g., 1 hour) at 5-30 Hz (e.g., 30 Hz) using a mill (e.g., Retsch Mixer Miller MM300). In some embodiments, samples initially ground with one solvent (e.g., acetonitrile or nitromethane) are dried, wetted with another solvent (e.g., n-heptane) and ground for about 0.5-5 hours (e.g., 1 hour) using the same conditions. All samples are then analysed (e.g., by XRPD).

In one of the embodiments, a mixture of obeticholic acid (about 30 mg) and coformer (e.g., UDCA) (about 1.1 eq) is placed in a stainless steel grinding jar (e.g., 2 mL) with one grinding ball (e.g., 7 mm grinding ball). The materials are wetted with acetonitrile (about 10 μL), nitromethane (about 20 μL) or heptane (about 10 μL) and ground for about 1 h at 30 Hz using a Retsch Mixer Miller MM300. Samples initially ground with acetonitrile or nitromethane are dried, wetted with about 10 μL of n-heptane and ground for about 1 hour using the above conditions. All samples are then analysed by XRPD. In one of the embodiments, the solvent is acetonitrile.

In some embodiments, a solution of obeticholic acid in a solvent (e.g., tetrahydrofuran or acetonitrile) is added to a neat coformer (e.g., UDCA) solid equal molar amount (e.g., about 1.1 eq). The samples are heated at about 20-70° C. (e.g., 50° C.) for about 10-90 minutes (e.g., 30 minutes) and cooled to about 10-0° C. (e.g., 5° C.) at about 1-0.05° C./min (e.g., 0.1° C./min) under stirring with a stirring speed of about 200-600 rpm (e.g., 300 rpm). In certain embodiments, the samples are filtered after the cooling regime. In certain embodiments, the samples are stirred at about 25-50° C. then cooled down to 20-25° C. (e.g., 25° C.) over 4- to 10-hour cycle (e.g., about 8 h cycle) for about 2 to 10 days (e.g., 5 days) prior to filtration. In certain embodiments, solutions obtained after cooling are treated with another solvent (e.g., heptane) and stirred at about 25-50° C. (8 h cycle) for about 2-10 days (e.g., 7 days). In one of the embodiments, the samples are further left to stand at ambient conditions for up to 2-10 days (e.g., 5 days). Solids obtained are analysed (e.g., by XRPD). In one of the embodiments, the solvent is acetonitrile. In one of the embodiments, an aliquot of a stock solution of obeticholic acid in acetonitrile is added to neat coformer solid (e.g., UDCA) (1.1 eq). The samples are heated at 50° C. for 30 minutes and cooled to 5° C. at 0.1° C./minute. Stir speed of about 300 rpm is typically maintained throughout the experiments. Some samples are filtered after the cooling regime. Some samples can be stirred at about 25-50° C. (8 h cycle) for about 1-5 days prior to filtration. Obtained solids are analysed by XRPD.

While not intending to be bound by any particular theory, certain solid forms provided herein exhibit physical properties, e.g., stability, solubility and/or dissolution rate, appropriate for use in clinical and therapeutic dosage forms. Moreover, while not wishing to be bound by any particular theory, certain solid forms provided herein exhibit physical properties, e.g., crystal morphology, compressibility and/or hardness, suitable for manufacture of a solid dosage form. In some embodiments, such properties can be determined using techniques such as X-ray diffraction, microscopy, IR spectroscopy and thermal analysis, as described herein and known in the art.

The cocrystaline forms can lead to enhancement of physical properties of the resulting solid forms, such as solubility, dissolution rate, bioavailablity, physical stability, chemical stability, flowability, fractability, or compressibility. Cocrystalline forms may be formed with many different counter-molecules, and some of these cocrystals may exhibit enhanced solubility or stability. Pharmaceutical cocrystals can increase the bioavailability or stability profile of a compound without the need for chemical (covalent) modification of the active pharmaceutical ingredient (API). Certain embodiments of the present disclosure relate to the cocrystalline forms of OCA which are stable on storage at elevated conditions, e.g. high humidity (e.g., 40° C./75% RH and 25° C./97% RH). The stability of cocrystalline forms of the present invention can be confirmed by unchanged peaks attributable to the cocrystal by XRPD performed after the storage period (e.g. 7 days).

In one of the embodiments, OCA-UDCA cocrystal is stable post 7 days of storage at elevated conditions (40° C./75% RH and 25° C./97% RH). The stability of cocrystalline form of obeticholic acid and ursodeoxycholic acid may be characterized by having unchanged XRPD peaks as shown in FIG. 18.

In some embodiments, the cocrystalline forms of OCA are stable at a humidity higher than about 60% RH (e.g., higher than about 70% RH, about 75% RH, about 80% RH, about 85% RH, about 90% RH, about 95% RH). In one embodiment, the cocrystalline form of OCA and UDCA is thermally stable at about 97% RH. In some embodiments, the cocrystalline form of OCA and UDCA is thermally stable at 40° C. In some embodiments, the cocrystalline form of OCA and UDCA is thermally stable at 40° C. and about 75% RH. In another embodiment, the cocrystalline form of OCA and UDCA is thermally stable at 25° C. and about 97% RH.

### Pharmaceutical Compositions

The present application addresses a need for new compositions of OCA, including crystalline forms of OCA (e.g., cocrystalline forms of OCA), and methods of preparing and using such compositions, formulations and dosage forms, to potentially permit, inter alia, convenient administration to patients, limited amount of impurities upon storage, suitable impurity profile to minimize potential toxicity, accurate delivery of intended dose, development of improved treatment regimens that maximize biologic activity, use of OCA solid forms for treating new diseases or disorders or new patient populations; and/or other potential benefits.

In some aspects, the present disclosure pertains to a pharmaceutical composition comprising the cocrystalline form of OCA and a co-former and a pharmaceutically acceptable diluent, excipient or carrier.

Some of the embodiments of the present disclosure pertains to a pharmaceutical composition comprising the cocrystalline form of OCA and a bile acid co-former and a pharmaceutically acceptable diluent, excipient or carrier.

In one of the embodiments, the present disclosure pertains to a pharmaceutical composition comprising the cocrystalline form of OCA and UDCA and a pharmaceutically acceptable diluent, excipient or carrier.

The present application provides pharmaceutical compositions comprising a crystalline form of obeticholic acid (e.g., cocrystal of OCA-UDCA), and a pharmaceutically acceptable diluent, excipient, or carrier. The pharmaceutical composition of the present disclosure can be administered enternally, orally, transdermally, pulmonarily, inhalationally, buccally, sublingually, intraperintoneally, subcutaneously, intramuscularly, intravenously, rectally, intrapleurally, intrathecally, intranasally, parenterally, or topically.

In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

tablets: mixing of active ingredient/sand auxiliaries, compression of said mixture into tablets (direct compression), optionally granulation of part of mixture before compression.

capsules: mixing of active ingredient/s and auxiliaries to obtain a flowable powder, optionally granulating powder, filling powders/granulate into opened capsules, capping of capsules.

semi-solids (ointments, gels, creams): dissolving/dispersing active ingredient/s in an aqueous or fatty carrier; subsequent mixing of aqueous/fatty phase with complementary fatty/aqueous phase, homogenization (creams only).

suppositories (rectal and vaginal): dissolving/dispersing active ingredient/sin carrier material liquified by heat (rectal: carrier material normally a wax; vaginal: carrier normally a heated solution of a gelling agent), casting said mixture into suppository forms, annealing and withdrawal suppositories from the forms.

aerosols: dispersing/dissolving active agent/sin a propellant, bottling said mixture into an atomizer.

Suitable formulations for parenteral administration include aqueous solutions of the active compounds in watersoluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances, which increase the viscosity of the suspension, including, for example, sodium carboxymethyl cellulose, sorbitol, and/or dextran, optionally, the suspension may also contain stabilizers. For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers. In addition, stabilizers may be added.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Dosage forms for the topical or transdermal administration include but are not limited to powders, sprays, ointments, pastes, creams, lotions, gels, solutions, patches and inhalants. In one embodiment, the active ingredient is mixed under sterile conditions with a pharmaceutically acceptable carrier, and with any preservatives, buffers or propellants that are required.

Suitable excipients are organic or inorganic substances, which are suitable for enteral (for example oral), parenteral or topical administration and do not react with the products of the disclosure, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The compounds of the disclosure can be used, for example, for the production of injection preparations. The preparations indicated can be sterilized and/or can contain excipients such as lubricants, preservatives, stabilizers and/or wetting agents, emulsifiers, salts for affecting the osmotic pressure, buffer substances, colorants, flavourings and/or aromatizers. They can, if desired, also contain one or more further active compounds, e.g. one or more vitamins.

For administration by inhalation, the active ingredient is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active ingredient is formulated into ointments, salves, gels, or creams as generally known in the art.

One skilled in the art will appreciate that it is sometimes necessary to make routine variations to the dosage depending on, for example, the age and condition of the patient. The dosage will also depend on the route of administration.

One skilled in the art will recognize the advantages of certain routes of administration. The dosage administered will be dependent upon the age, health, and weight of recipient, kind of concurrent treatment, if any, frequency of treatment, and the nature of the effect desired.

In one embodiment, the pharmaceutical composition of the present application is administered orally.

Oral compositions generally include an inert diluent or an edible pharmaceutically acceptable carrier. They can be enclosed in gelatin capsules or compressed into tablets. For the purpose of oral therapeutic administration, the active ingredient can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures.

In order to produce dosage form coatings resistant to gastric juices or to provide a dosage form affording the advantage of prolonged action (modified release dosage form), the tablet, dragee or pill can comprise an inner dosage and an outer dosage component me latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses. Suitable carrier substances are organic or inorganic substances which are suitable for enteral (e.g. oral) or parenteral administration or topical application and do not react with the compounds of disclosure, for example water, vegetable oils, benzyl alcohols, polyethylene glycols, gelatin, carbohydrates such as lactose or starch, magnesium stearate, talc and petroleum jelly.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin.

The liquid forms in which the compositions of the present disclosure may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Dosage forms for oral administration comprise modified release formulations. The term “immediate release” is defined as a release of the disclosed crystalline form of obeticholic acid (e.g., cocrystal of OCA-UDCA) from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of the disclosed crystalline form of obeticholic acid (e.g., cocrystal of OCA-UDCA) from a dosage form over a prolonged period of time.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active ingredient calculated to produce the desired therapeutic effect in association with the required pharmaceutical carrier. The specification for the dosage unit forms of the application are dictated by and directly dependent on the unique characteristics of the active ingredient and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Dosages can range from about 0.01 mg/kg per day to about 500 mg/kg of the disclosed crystalline form of obeticholic acid (e.g., cocrystal of OCA-UDCA) per day. In one of the embodiments, the daily dose is preferably between about 0.01 mg/kg and 10 mg/kg of body weight.

Those of skill will readily appreciate that in one of the embodiments, the composition or formulation comprises about 0.1 mg to about 1500 mg of the disclosed crystalline form of obeticholic acid (e.g., cocrystal of OCA-UDCA) per dosage form. In another embodiment, the formulation or composition comprises about 1 mg to about 100 mg of the disclosed crystalline form of obeticholic acid (e.g., cocrystal of OCA-UDCA). In another embodiment, the formulation comprises about 1 mg to about 50 mg. In another embodiment, the formulation comprises about 1 mg to about 30 mg. In another embodiment, the formulation comprises about 4 mg to about 26 mg. In another embodiment, the formulation comprises about 5 mg to about 25 mg. In one embodiment, the formulation comprises about 1 mg to about 5 mg. In one embodiment, the formulation comprises about 1 mg to about 2 mg.

An effective amount of a pharmaceutical agent is that which provides an objectively identifiable improvement as noted by the clinician or other qualified observer.

The pharmaceutical compositions can be included in a container, kit, pack, or dispenser together with instructions for administration.

The pharmaceutical compositions containing the disclosed crystalline forms of obeticholic acid may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active ingredient into preparations that can be used pharmaceutically. Of course, the appropriate formulation is dependent upon the route of administration chosen.

Techniques for formulation and administration of the disclosed crystalline forms or obeticholic acid can be found in Remington: The Science and Practice of Pharmacy, 19th edition, Mack Publishing Co., Easton, Pa. (1995) or any later versions thereof.

The active ingredient can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers.

### Methods of the Application

Some aspects of the present disclosure pertain to a method of treating or preventing a varierty of liver, metabolic, kidney, cardiovascular, gastrointestinal and cancerous diseases, disorders or conditions. In some embodiments, the present disclosure relates to a method of treating or preventing an FXR-mediated disease or disorder in a subject in need thereof, comprising administering a therapeutically effective amount of the crystalline form of OCA. In some embodiments the crystalline form of obeticholic acid is a cocrystal of OCA and a coformer. In some embodiments, the coformer is a bile acid. In one of the embodiments, the coformer is UDCA.

It is well known that natural bile acids and bile acid derivatives modulate not only nuclear hormone receptors, but are also modulators for the G protein-coupled receptor (GPCR) TGR5. In some aspects, this application pertains to a crystalline form of obeticholic acid (e.g., cocrystal of OCA-UDCA) or a pharmaceutical composition comprising a crystalline form of obeticholic acid (e.g., cocrystal of OCA-UDCA) for treating or preventing or modulating an TGR5-mediated disease or disorder.

In some embodiments, the present disclosure relates to a method of treating or preventing an TGR5-mediated disease or disorder in a subject in need thereof, comprising administering a therapeutically effective amount of the crystalline form of OCA. In some embodiments the crystalline form of obeticholic acid is a cocrystal of OCA and a coformer. In some embodiments, the coformer is a bile acid. In one of the embodiments, the coformer is UDCA.

Some aspects of the present disclosure pertain to a method of modulating FXR or TGR5 activity in a subject in need thereof, comprising administering a therapeutically effective amount of the crystalline form of OCA. Some aspects of the present disclosure pertain to a method of modulating FXR or TGR5 activity in a subject in need thereof, comprising administering a therapeutically effective amount of the cocrystalline form of OCA and a co-former. In some embodiments the co-former is a bile acid. In one of the embodiments, the co-former is UDCA.

In certain embodiments, this disclosure pertains to a method of treating or preventing an FXR- or TGR5-mediated condition, disease or disorder in a subject in need thereof, comprising administering a therapeutically effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one aspect, this application pertains to a crystalline form of obeticholic acid (e.g., cocrystal of OCA-UDCA) or a pharmaceutical composition comprising a crystalline form of obeticholic acid (e.g., cocrystal of OCA-UDCA) for treating or preventing an FXR-mediated disease or disorder.

In one aspect, this application pertains to the use of a crystalline form of obeticholic acid (e.g., cocrystal of OCA-UDCA) or a pharmaceutical composition comprising a crystalline form of obeticholic acid (e.g., cocrystal of OCA-UDCA) in the manufacture of a medicament for treating or preventing a disease or disorder in which FXR plays a role.

In one embodiment, the disclosure relates to a method of treating or preventing chronic liver disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating chronic liver disease. In one embodiment, the disclosure relates to a method of preventing chronic liver disease. In one embodiment, the chronic liver disease is selected from primary biliary cirrhosis (also known as primary biliary cholangitis or PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, and alpha 1-antitrypsin deficiency.

In one embodiment, the disclosure relates to a method of treating or preventing one or more symptoms of cholestasis, including complications of cholestasis in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating one or more symptoms of cholestasis. In one embodiment, the disclosure relates to preventing one or more symptoms of cholestasis.

Cholestasis is typically caused by factors within the liver (intrahepatic) or outside the liver (extrahepatic) and leads to the accumulation of bile salts, bile pigment bilirubin, and lipids in the blood stream instead of being eliminated normally. Intrahepatic cholestasis is characterized by widespread blockage of small ducts or by disorders, such as hepatitis, that impair the body's ability to eliminate bile. Intrahepatic cholestasis may also be caused by alcoholic liver disease, primary biliary cirrhosis, cancer that has spread (metastasized) from another part of the body, primary sclerosing cholangitis, gallstones, biliary colic, and acute cholecystitis. It can also occur as a complication of surgery, serious injury, cystic fibrosis, infection, or intravenous feeding or be drug induced. Cholestasis may also occur as a complication of pregnancy and often develops during the second and third trimesters.

Extrahepatic cholestasis is most often caused by choledocholithiasis (Bile Duct Stones), benign biliary strictures (non-cancerous narrowing of the common duct), cholangiocarcinoma (ductal carcinoma), and pancreatic carcinoma. Extrahepatic cholestasis can occur as a side effect of many medications.

A crystalline form of obeticholic acid (e.g., cocrystal of OCA-UDCA) or a pharmaceutical composition comprising a crystalline form of obeticholic acid (e.g., cocrystal of OCA-UDCA) may be used for treating or preventing one or more symptoms of intrahepatic or extrahepatic cholestasis, including without limitation, biliary atresia, obstetric cholestasis, neonatal cholestasis, drug induced cholestasis, cholestasis arising from Hepatitis C infection, chronic cholestatic liver disease such as primary biliary cirrhosis (PBC), and primary sclerosing cholangitis (PSC).

In one embodiment, the disclosure relates to a method of enhancing liver regeneration in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the method is enhancing liver regeneration for liver transplantation.

In one embodiment, the disclosure relates to a method of treating or preventing fibrosis in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating fibrosis. In one embodiment, the disclosure relates to a method of preventing fibrosis.

Accordingly, as used herein, the term fibrosis refers to all recognized fibrotic disorders, including fibrosis due to pathological conditions or diseases, fibrosis due to physical trauma (“traumatic fibrosis”), fibrosis due to radiation damage, and fibrosis due to exposure to chemotherapeutics. As used herein, the term “organ fibrosis” includes but is not limited to liver fibrosis, fibrosis of the kidneys, fibrosis of lung, and fibrosis of the intestine. “Traumatic fibrosis” includes but is not limited to fibrosis secondary to surgery (surgical scarring), accidental physical trauma, burns, and hypertrophic scarring.

As used herein, “liver fibrosis” includes liver fibrosis due to any cause, including but not limited to virally-induced liver fibrosis such as that due to hepatitis B or C virus; exposure to alcohol (alcoholic liver disease), certain pharmaceutical compounds including but not limited to methotrexate, some chemotherapeutic agents, and chronic ingestion of arsenicals or vitamin A in megadoses, oxidative stress, cancer radiation therapy or certain industrial chemicals including but not limited to carbon tetrachloride and dimethylnitrosamine; and diseases such as primary biliary cirrhosis, primary sclerosing cholangitis, fatty liver, obesity, non-alcoholic steatohepatitis, cystic fibrosis, hemochromatosis, auto-immune hepatitis, and steatohepatitis. Current therapy in liver fibrosis is primarily directed at removing the causal agent, e.g., removing excess iron (e.g., in the case of hemochromatosis), decreasing viral load (e.g., in the case of chronic viral hepatitis), or eliminating or decreasing exposure to toxins (e.g., in the case of alcoholic liver disease). Anti-inflammatory drugs such as corticosteroids and colchicine are also known for use in treating inflammation that can lead to liver fibrosis. As is known in the art, liver fibrosis may be clinically classified into five stages of severity (S0, S1, S2, S3, and S4), usually based on histological examination of a biopsy specimen. S0 indicates no fibrosis, whereas S4 indicates cirrhosis. While various criteria for staging the severity of liver fibrosis exist, in general early stages of fibrosis are identified by discrete, localized areas of scarring in one portal (zone) of the liver, whereas later stages of fibrosis are identified by bridging fibrosis (scarring that crosses zones of the liver).

In one embodiment, the disclosure relates to a method of treating or preventing organ fibrosis in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the fibrosis is liver fibrosis.

In some embodiments, the liver disease or disorder is a chronic liver disease which can be primary biliary cirrhosis (also known in the art as primary biliary cholangitis or PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency.

In one of the embodiments, the metabolic disease is insulin resistance, Type I and Type II diabetes, or obesity.

In one of the embodiments, the renal disease is diabetic nephropathy, focal segmental glomerulosclerosis (FSGS), hypertensive nephrosclerosis, chronic glomerulonephritis, chronic transplant glomerulopathy, chronic interstitial nephritis, or polycystic kidney disease. In some embodiments, the disclosure relates to a method of treating or preventing cardiovascular disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the invention relates to a method of treating cardiovascular disease. In one embodiment, cardiovascular disease selected from atherosclerosis, arteriosclerosis, dyslipidemia, hypercholesteremia, hyperlipidemia, hyperlipoproteinemia, and hypertriglyceridemia.

The term “hyperlipidemia” refers to the presence of an abnormally elevated level of lipids in the blood. Hyperlipidemia can appear in at least three forms: (1) hypercholesterolemia, i.e., an elevated cholesterol level; (2) hypertriglyceridemia, i.e., an elevated triglyceride level; and (3) combined hyperlipidemia, i.e., a combination of hypercholesterolemia and hypertriglyceridemia.

The term “dyslipidemia” refers to abnormal levels of lipoproteins in blood plasma including both depressed and/or elevated levels of lipoproteins (e.g., elevated levels of LDL, VLDL and depressed levels of HDL).

In one embodiment, the disclosure relates to a method selected from reducing cholesterol levels or modulating cholesterol metabolism, catabolism, absorption of dietary cholesterol, and reverse cholesterol transport in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA).

In another embodiment, the disclosure relates to a method of treating or preventing a disease affecting cholesterol, triglyceride, or bile acid levels in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one embodiment, the disclosure relates to a method of lowering triglycerides in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one embodiment, the disclosure relates to a method of preventing a disease state associated with an elevated cholesterol level in a subject. In one embodiment, the disease state is selected from coronary artery disease, angina pectoris, carotid artery disease, strokes, cerebral arteriosclerosis, and xanthoma.

In one embodiment, the disclosure relates to a method of treating or preventing a lipid disorder in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating a lipid disorder. In one embodiment, the disclosure relates to a method of preventing a lipid disorder.

Lipid disorders are the term for abnormalities of cholesterol and triglycerides. Lipid abnormalities are associated with an increased risk for vascular disease, and especially heart attacks and strokes. Abnormalities in lipid disorders are a combination of genetic predisposition as well as the nature of dietary intake. Many lipid disorders are associated with being overweight. Lipid disorders may also be associated with other diseases including diabetes, the metabolic syndrome (sometimes called the insulin resistance syndrome), underactive thyroid or the result of certain medications (such as those used for anti-rejection regimens in people who have had transplants).

In one embodiment, the disclosure relates to a method of treating or preventing one or more symptoms of disease affecting lipid metabolism (i.e., lipodystrophy) in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating one or more symptoms of a disease affecting lipid metabolism. In one embodiment, the disclosure relates to a method of preventing one or more symptoms of a disease affecting lipid metabolism.

In one embodiment, the disclosure relates to a method of decreasing lipid accumulation in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one embodiment, the disclosure relates to a method of treating or preventing gastrointestinal disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating gastrointestinal disease. In one embodiment, the disclosure relates to a method of preventing gastrointestinal disease. In one embodiment, the gastrointestinal disease is selected from inflammatory bowel disease (IBD), irritable bowel syndrome (IBS), bacterial overgrowth, malabsorption, post-radiation colitis, and microscopic colitis. In one embodiment, the inflammatory bowel disease is selected from Crohn's disease and ulcerative colitis.

In one embodiment, the disclosure relates to a method of treating or preventing renal disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one embodiment, the disclosure relates to a method of treating renal disease. In one embodiment, the invention relates to a method of preventing renal disease. In one embodiment, the renal disease is selected from diabetic nephropathy, focal segmental glomerulosclerosis (FSGS), hypertensive nephrosclerosis, chronic glomerulonephritis, chronic transplant glomerulopathy, chronic interstitial nephritis, and polycystic kidney disease.

In one embodiment, the disclosure relates to a method of treating or preventing metabolic disease in a subject, comprising administering to the subject in need thereof an effective amount a crystalline form of obeticholic acid (e.g., cocrystal of OCA-UDCA) or a pharmaceutical composition comprising a crystalline form of obeticholic acid (e.g., cocrystal of OCA-UDCA. In one embodiment, the metabolic disease is selected from insulin resistance, hyperglycemia, diabetes mellitus, diabesity, and obesity. In one embodiment, the diabetes mellitus is type I diabetes. In one embodiment, the diabetes mellitus is type II diabetes.

Diabetes mellitus, commonly called diabetes, refers to a disease or condition that is generally characterized by metabolic defects in production and utilization of glucose which result in the failure to maintain appropriate blood sugar levels in the body.

In the case of type II diabetes, the disease is characterized by insulin resistance, in which insulin loses its ability to exert its biological effects across a broad range of concentrations. This resistance to insulin responsiveness results in insufficient insulin activation of glucose uptake, oxidation and storage in muscle and inadequate insulin repression of lipolysis in adipose tissue and of glucose production and secretion in liver. The resulting condition is elevated blood glucose, which is called “hyperglycemia”. Uncontrolled hyperglycemia is associated with increased and premature mortality due to an increased risk for microvascular and macrovascular diseases, including retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys), hypertension, cerebrovascular disease, and coronary heart disease. Therefore, control of glucose homeostasis is a critically important approach for the treatment of diabetes.

Insulin resistance has been hypothesized to unify the clustering of hypertension, glucose intolerance, hyperinsulinemia, increased levels of triglyceride and decreased HDL cholesterol, and central and overall obesity. The association of insulin resistance with glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, has been referred to as “Syndrome X”. Accordingly, methods of treating or preventing any disorders related to insulin resistance including the cluster of disease states, conditions or disorders that make up “Syndrome X” are provided. In one embodiment, the invention relates to a method of treating or preventing metabolic syndrome in a subject, comprising administering to the subject in need thereof an effective amount of a compound of the invention or a pharmaceutically acceptable salt, solvate, or amino acid conjugate thereof. In one embodiment, the invention relates to a method of treating metabolic syndrome. In another embodiment, the invention relates to a method of preventing metabolic syndrome.

In one embodiment, the disclosure relates to a method of treating or preventing cancer in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating cancer. In one embodiment, the disclosure relates to a method of preventing cancer. In one embodiment, the cancer is selected from hepatocellular carcinoma, colorectal cancer, gastric cancer, renal cancer, prostate cancer, adrenal cancer, pancreatic cancer, breast cancer, bladder cancer, salivary gland cancer, ovarian cancer, uterine body cancer, and lung cancer. In one embodiment, the cancer is hepatocellular carcinoma. In one embodiment, the cancer is colorectal cancer. In one embodiment, the cancer is gastric cancer. In one embodiment, the cancer is renal cancer. In one embodiment, the cancer is prostate cancer. In one embodiment, the cancer is adrenal cancer. In one embodiment, the cancer is pancreatic cancer. In one embodiment, the cancer is breast cancer. In one embodiment, the cancer is bladder cancer. In one embodiment, the cancer is salivary gland cancer. In one embodiment, the cancer is ovarian cancer. In one embodiment, the cancer is uterine body cancer. In one embodiment, the cancer is lung cancer.

In another embodiment, at least one of an agent selected from Sorafenib, Sunitinib, Erlotinib, or Imatinib is co-administered with the crystalline form of the present disclosure to treat cancer. In one embodiment, at least one of an agent selected from abarelix, aldeleukin, allopurinol, altretamine, amifostine, anastozole, bevacizumab, capecitabine, carboplatin, cisplatin, docetaxel, doxorubicin, erlotinib, exemestane, 5-fluorouracil, fulvestrant, gemcitabine, goserelin acetate, irinotecan, lapatinib ditosylate, letozole, leucovorin, levamisole, oxaliplatin, paclitaxel, panitumumab, pemetrexed disodium, profimer sodium, tamoxifen, topotecan, and trastuzumab is co-administered with the compound of the invention to treat cancer.

Appropriate treatment for cancers depends on the type of cell from which the tumor derived, the stage and severity of the malignancy, and the genetic abnormality that contributes to the tumor.

Cancer staging systems describe the extent of cancer progression. In general, the staging systems describe how far the tumor has spread and puts patients with similar prognosis and treatment in the same staging group. In general, there are poorer prognoses for tumors that have become invasive or metastasized.

In one type of staging system, cases are grouped into four stages, denoted by Roman numerals I to IV. In stage I, cancers are often localized and are usually curable. Stage II and IIIA cancers are usually more advanced and may have invaded the surrounding tissues and spread to lymph nodes. Stage IV cancers include metastatic cancers that have spread to sites outside of lymph nodes.

Another staging system is TNM staging which stands for the categories: Tumor, Nodes, and Metastases. In this system, malignancies are described according to the severity of the individual categories. For example, T classifies the extent of a primary tumor from 0 to 4 with 0 representing a malignancy that does not have invasive activity and 4 representing a malignancy that has invaded other organs by extension from the original site. N classifies the extent of lymph node involvement with 0 representing a malignancy with no lymph node involvement and 4 representing a malignancy with extensive lymph node involvement. M classifies the extent of metastasis from 0 to 1 with 0 representing a malignancy with no metastases and 1 representing a malignancy with metastases.

These staging systems or variations of these staging systems or other suitable staging systems may be used to describe a tumor such as hepatocellular carcinoma. Few options only are available for the treatment of hepatocellular cancer depending on the stage and features of the cancer. Treatments include surgery, treatment with Sorafenib, and targeted therapies. In general, surgery is the first line of treatment for early stage localized hepatocellular cancer. Additional systemic treatments may be used to treat invasive and metastatic tumors.

In one embodiment, the disclosure relates to a method of treating or preventing gallstones in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating gallstones. In one embodiment, the disclosure relates to a method of preventing gallstones.

A gallstone is a crystalline concretion formed within the gallbladder by accretion of bile components. These calculi are formed in the gallbladder but may distally pass into other parts of the biliary tract such as the cystic duct, common bile duct, pancreatic duct, or the ampulla of Vater. Rarely, in cases of severe inflammation, gallstones may erode through the gallbladder into adherent bowel potentially causing an obstruction termed gallstone ileus. Presence of gallstones in the gallbladder may lead to acute cholecystitis, an inflammatory condition characterized by retention of bile in the gallbladder and often secondary infection by intestinal microorganisms, predominantly Escherichia coli, and Bacteroides species. Presence of gallstones in other parts of the biliary tract can cause obstruction of the bile ducts, which can lead to serious conditions such as ascending cholangitis or pancreatitis.

In one embodiment, the disclosure relates to a method of treating or preventing cholesterol gallstone disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating cholesterol gallstone disease. In one embodiment, the disclosure relates to a method of preventing cholesterol gallstone disease.

In one embodiment, the disclosure relates to a method of treating or preventing neurological disease in a subject, comprising administering to the subject in need thereof an effective amount of a crystalline form of obeticholic acid (e.g., cocrystal of OCA-UDCA) or a pharmaceutical composition comprising a crystalline form of obeticholic acid (e.g., cocrystal of OCA-UDCA). In one embodiment, the disclosure relates to a method of treating neurological disease. In one embodiment, the disclosure relates to a method of preventing neurological disease. In one embodiment, the neurological disease is stroke.

In one embodiment, the disclosure relates to a method of regulating the expression level of one or more genes involved in bile acid homeostasis.

In one embodiment, the disclosure relates to a method of down regulating the expression level of one or more genes selected from CYP7α1 and SREBP-IC in a cell by administering to the cell a crystalline form of OCA. In one embodiment, the disclosure relates to a method of up regulating the expression level of one or more genes selected from OSTα, OSTβ, BSEP, SHP, UGT2B4, MRP2, FGF-19, PPARγ, PLTP, APOCII, and PEPCK in a cell by administering to the cell a crystalline form of the invention.

The amount of the crystalline form of obeticholic acid (e.g., cocrystal of OCA-UDCA) which is required to achieve the desired biological effect will depend on a number of factors such as the use for which it is intended, the means of administration, and the recipient, and will be ultimately at the discretion of the attendant physician or veterinarian. In general, a typical daily dose for the treatment of a FXR mediated disease and condition, for instance, may be expected to lie in the range of from about 0.01 mg/kg to about 100 mg/kg. This dose may be administered as a single unit dose or as several separate unit doses or as a continuous infusion. Similar dosages would be applicable for the treatment of other diseases, conditions and therapies including the prevention and treatment of cholestatic liver diseases.

In some of the embodiments, the gastrointestinal disease is inflammatory bowel disease (IBD) (including Crohn's disease and ulcerative colitis), irritable bowel syndrome (IBS), bacterial overgrowth, malabsorption, post-radiation colitis, or microscopic colitis.

In one aspect, this application pertains to a method of modulating FXR (e.g., activating FXR) in a subject in need thereof, comprising administering a therapeutically effective amount of a crystalline form of obeticholic acid (e.g., cocrystal of OCA-UDCA) or a pharmaceutical composition comprising a crystalline form of obeticholic acid (e.g., cocrystal of OCA-UDCA).

In one aspect, this application pertains to a method of modulating TGR5 (e.g., activating TGR5) in a subject in need thereof, comprising administering a therapeutically effective amount of a crystalline form of obeticholic acid (e.g., cocrystal of OCA-UDCA) or a pharmaceutical composition comprising a crystalline form of obeticholic acid (e.g., cocrystal of OCA-UDCA).

The disclosure also relates to the manufacture of a medicament for treating or preventing a disease or condition (e.g., a disease or condition mediated by FXR), wherein the medicament comprises a crystalline form of obeticholic acid (e.g., cocrystal of OCA-UDCA) or a pharmaceutical composition comprising a crystalline form of obeticholic acid (e.g., cocrystal of OCA-UDCA).

In one aspect, this application pertains to use of a crystalline form of obeticholic acid (e.g., cocrystal of OCA-UDCA) or a pharmaceutical composition comprising a crystalline form of obeticholic acid (e.g., cocrystal of OCA-UDCA) in the manufacture of a medicament for modulating FXR (e.g., activating FXR).

In one aspect, this application pertains to use of a crystalline form of obeticholic acid (e.g., cocrystal of OCA-UDCA) or a pharmaceutical composition comprising a crystalline form of obeticholic acid (e.g., cocrystal of OCA-UDCA) in the manufacture of a medicament for modulating TGR5 (e.g., activating TGR5).

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present disclosure the skilled artisan can identify and employ other components and methodology useful for practicing the present application.

### EXAMPLES

### Instrument and Methodology

### X-Ray Powder Diffraction (XRPD)

### Bruker AXS C2 GADDS

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gobel multilayer mirror coupled with a pinhole collimator of 0.3 mm. A weekly performance check is carried out using a certified standard NIST 1976 Corundum (flat plate).

The beam divergence, i.e., the effective size of the X-ray beam on the sample, was approximately 4 mm. A θ-θ (theta-theta) continuous scan mode was employed with a sample—detector distance of 20 cm which gives an effective 2θ (theta) range of about 3.2°-29.7°. Typically, the sample would be exposed to the X-ray beam for about 120 seconds. The software used for data collection was GADDS for XP/2000 4.1.43 and the data were analyzed and presented using Diffrac Plus EVA v15.0.0.0.

Ambient conditions: Samples run under ambient conditions were prepared as flat plate specimens using powder as received without grinding. Approximately 1-2 mg of the sample was lightly pressed on a glass slide to obtain a flat surface.

Non-ambient conditions: Samples run under non-ambient conditions were mounted on a silicon wafer with heat-conducting compound. The sample was then heated to the appropriate temperature at about 20° C./min and subsequently held isothermally for about 1 minute before data collection was initiated.

### Bruker AXS D8 Advance

X-Ray Powder Diffraction patterns were collected on a Bruker D8 diffractometer using Cu Kα radiation (40 kV, 40 mA), 0-2θ (theta) goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analyzed and presented using Diffrac Plus EVA v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are:
Angular range: 2 to 42° 2θ (theta)
Step size: 0.05° 2θ (theta)
Collection time: 0.5 s/step

### Single Crystal X-Ray Diffraction (SCXRD)

### Oxford Diffraction Supernova Dual Source Cu at Zero Atlas CCD

Data were collected on an Oxford Diffraction Supernova Dual Source, Cu at Zero, Atlas CCD diffractometer equipped with an Oxford Cryosystems Cobra cooling device. The data was collected using CuKα radiation. Structures were typically solved using either the SHELXS or SHELXD programs and refined with the SHELXL program as part of the Bruker AXS SHELXTL suite (V6.10). Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### Nuclear Magnetic Resonance (NMR)

### ¹H NMR

NMR spectra were collected on a Bruker 400 MHz instrument equipped with an auto-sampler and controlled by a DRX400 console. Automated experiments were acquired using ICON-NMR v4.0.7 running with Topspin v1.3 using the standard Bruker loaded experiments.

Samples were prepared in DMSO-d6, unless otherwise stated. Off-line analysis was carried out using ACD Spectrus Processor 2012.

### Fourier Transform—Infra-Red (FTIR)

Data were collected on a Perkin-Elmer Spectrum One fitted with a universal Attenuated Total Reflectance (ATR) sampling accessory. The data were collected and analyzed using Spectrum v10.0.1 software and off-line analysis was carried out using ACD Spectrus Processor 2012.

Differential Scanning calorimetry (DSC)

### TA Instruments Q2000

DSC data were collected on a TA Instruments Q2000 equipped with a 50 position auto-sampler. The calibration for thermal capacity was carried out using sapphire and the calibration for energy and temperature was carried out using certified indium. Typically, about 2-3 mg of each sample, in a pin-holed aluminum pan, was heated at about 10° C./min from about 25° C. to about 200° C. A purge of dry nitrogen at about 50 mL/min was maintained over the sample. The instrument control software was Advantage for Q Series v2.8.0.394 and Thermal Advantage v5.5.3 and the data were analysed using Universal Analysis v4.5A.

### Mettler DSC 823e

DSC data were collected on a Mettler DSC 823E equipped with a 34 position auto-sampler. The instrument was calibrated for energy and temperature using certified indium. Typically, about 0.5-5 mg of each sample, in a pin-holed aluminium pan, was heated at about 10° C./min from about 25° C. to about 350° C. A nitrogen purge at about 50 ml/min was maintained over the sample. The instrument control and data analysis software was STARe v12.1.

### Thermo-Gravimetric Analysis (TGA)

TGA data were collected on a TA Instruments Q500 TGA, equipped with a 16 position autosampler. The instrument was temperature calibrated using certified Alumel and Nickel. Typically, about 2-11 mg of each sample was loaded onto a pre-tared aluminium DSC pan and heated at about 10° C./min from ambient temperature to about 350° C. A nitrogen purge at about 60 ml/min was maintained over the sample. The instrument control software was Advantage for Q Series v2.5.0.256 and Thermal Advantage v5.5.3 and the data were analysed using Universal Analysis v4.5A.

### Polarized Light Microscopy (PLM)

### Leica LM/DM Polarised Light Microscope

Samples were studied on a Leica LM/DM polarized light microscope with a digital video camera for image capture. A small amount of each sample was placed on a glass slide, mounted in immersion oil and covered with a glass slip, the individual particles being separated as well as possible. The sample was viewed with appropriate magnification and partially polarized light, coupled to a λ false-color filter.

### Hot Stage Microscopy (HSM)

Hot Stage Microscopy was carried out using a Leica LM/DM polarised light microscope combined with a Mettler-Toledo FP82HT hot-stage and a digital video camera for image capture. A small amount of each sample was placed onto a glass slide with individual particles separated as well as possible. The sample was viewed with appropriate magnification and partially polarised light, coupled to a λ false-colour filter, whilst being heated from ambient temperature typically at about 10-20° C./min.

### Water Determination by Karl Fischer Titration (KF)

The water content of each sample was measured on a Metrohm 874 Oven Sample Processor at about 150° C. with 851 Titrano Coulometer using Hydranal Coulomat AG oven reagent and nitrogen purge. Weighed solid samples were introduced into a sealed sample vial. Approximately 10 mg of sample was used per titration and duplicate determinations were made. Data collection and analysis were carried out using Tiamo v2.2.

### Gravimetric Vapor Sorption (GVS)

Sorption isotherms were obtained using a SMS DVS Intrinsic moisture sorption analyzer, controlled by DVS Intrinsic Control software v1.0.1.2 (or v 1.0.1.3). The sample temperature was maintained at about 25° C. by the instrument controls. The humidity was controlled by mixing streams of dry and wet nitrogen, with a total flow rate of about 200 mL/min. The relative humidity was measured by a calibrated Rotronic probe (dynamic range of about 1.0-100% RH), located near the sample. The weight change, (mass relaxation) of the sample as a function of % RH was constantly monitored by the microbalance (accuracy ±0.005 mg). Typically, about 18-20 mg of sample was placed in a tared mesh stainless steel basket under ambient conditions. The sample was loaded and unloaded at about 40% RH and about 25° C. (typical room conditions). A moisture sorption isotherm was performed as outlined in Table 1 (2 scans giving 1 complete cycle). The standard isotherm was performed at about 25° C. at about 10% RH intervals over a 0-90% RH range. Data analysis was carried out using Microsoft Excel using DVS Analysis Suite v6.2 (or 6.1 or 6.0). The sample was recovered after completion of the isotherm and re-analysed by XRPD.

TABLE 1
Method for SMS DVS Intrinsic experiments

| Parameter | Value |
|---|---|
| Adsorption - Scan 1 | 40-90 |
| Desorption/Adsorption - Scan 2 | 90-0, 0-40 |
| Intervals (% RH) | 10 |
| Number of Scans | 2 |
| Flow rate (mL/min) | 200 |
| Temperature (° C.) | 25 |
| Stability (° C./min) | 0.2 |
| Sorption Time (hours) | 6 hour time out |

### Ion Chromatography (IC)

Data were collected on a Metrohm 761 Compact IC (for cations) using IC Net software v2.3. Accurately weighed samples were prepared as stock solutions in an appropriate dissolving solution and diluted appropriately prior to testing. Quantification was achieved by comparison with standard solutions of known concentration of the ion being analyzed.

TABLE 2
IC method for cation chromatography

| Parameter | Value |
|---|---|
| Type of method | Cation exchange |
| Column | Metrosep C 4 - 250 (4.0 × 250 mm) |
| Column Temperature (° C.) | Ambient |
| Injection (μl) | 10 |
| Detection | Conductivity detector |
| Flow Rate (mL/min) | 1.0 |
| Eluent | 1.7 mM Nitric Acid |
| | 0.7 mM Dipicolinic acid in a 5% |
| | acetone aqueous solution. |

### Example 1. Salt Screening Procedures

Cooling with Acetonitrile and Isopropyl Alcohol (IPA)

Obeticholic acid (about 30 mg) was dissolved in acetonitrile (about 0.9 mL) at about 50° C. and about 1.1 eq of counter-ion solution was added. After precipitation, the samples were cooled down to about 5° C. at about 1° C./min and stirred at this temperature for about one hour. Experiments with a slower cooling rate of about 0.1° C./min from about 50° C. to about 5° C. were also performed.

Solids were filtered, dried under vacuum and analysed by XRPD.

The cooling procedure for screening in isopropyl alcohol (IPA) was performed as discussed above with about 0.15 mL of solvent. Solutions were divided into two portions for slow evaporation and anti-solvent addition experiments as discussed herein.

### Maturation at 25° C./50° C.

The amorphous samples from cooling experiments in acetonitrile were re-suspended in about 300 μL of acetonitrile. Samples were stirred at about 500 rpm at about 25 to about 50° C. (about 8 h cycle) for about 20 hours. Suspensions were dried for about 5 hours under vacuum (at about 25° C.) and analysed by XRPD. The solution obtained can be split into two portions for slow evaporation and anti-solvent addition experiments as discussed herein.

### Maturation at 50° C.

Obeticholic acid (about 30 mg) was dissolved in acetonitrile (about 0.9 mL) at about 50° C. and about 1.1 eq of counter-ion solution was added. Precipitation occurred after the counterion was added. The samples were left stirring at about 50° C. and about 250 rpm for about 24 hours. Suspensions were filtered, dried under vacuum for about 5 hours under vacuum (at about 25° C.) and analysed by XRPD.

### Slow Evaporation

Sample solutions (about 50 μL) from IPA cooling experiments were placed in sealed vials with a microneedle inserted through the lid to allow slow evaporation of solvent. Solutions obtained after maturation at about 25° C.-50° C. in acetonitrile (about 500 μL) was also subjected to slow evaporation. After about four weeks of slow evaporation, XRPD data was obtained for all solids.

### Antisolvent Addition

The remaining solutions, after an aliquot was taken for slow evaporation were held at about 50° C. for about 15 min and then treated with anti-solvent, e.g. water. The samples were cooled to about 5° C. at about 1° C./min while stirring at about 500 rpm. After cooling, samples

were left to evaporate to dryness under ambient conditions. XRPD data was obtained for powdered samples.

### Experiments Using Water/Heptane

Obeticholic acid (about 30 mg) was dissolved in aqueous counter-ion (about 1.1 eq) solution to make a final volume of about 0.16 mL. The samples were heated at about 50° C. for about 30 minutes and cooled to about 5° C. at about 0.1° C./minute. Stirring speed of about 300 rpm was maintained throughout the experiments. The samples that remained solutions were topped up with water (about 0.5 mL) and lyophilised. The lyophilised solids were suspended in heptane (about 0.6 mL) and left stirring at about 50° C. (at about 300 rpm) for about 5 days. Solids were filtered under partial vacuum (suction) Titration and analysed by XRPD.

### Example 2. Obeticholic Acid Monoammonium Salt—Form 1

Obeticholic acid (about 1000 mg) was treated with about 30 mL of acetonitrile and the sample was rapidly heated to about 50° C. After stirring for about 10 minutes at about 50° C., about 365 μL of a 7.2M ammonium hydroxide solution (about 1.1 eq) was added to the sample. The sample was left stirring at about 50° C. for about 22 hours at the stirring rate of about 300 rpm. The formed precipiate was filtered under suction filtration and dried under vacuum at about 35° C. for about 20 hours to afford about 942.8 mg of the product, obeticholic acid monoammonium salt.

### Characterisation of Obeticholic Acid Monoammonium Salt

XRPD diffractogram of the crystalline obeticholic acid monoammonium salt is shown in FIG. 1. Obeticholic acid monoammonium salt is also characterized by ¹H NMR as shown in FIG. 2. According to Polarized Light Microscopy analysis (PLM), the irregular particles of crystalline OCA monoamonium salt are less than 40 μm in length (see FIG. 9).

Thermal data is consistent with that obtained for the screening sample. The DSC endothermic events in the range from about 105-200° C. coincide with a TGA weight loss as shown in FIG. 3. The components lost during this weight loss appear integral to maintaining the crystal structure of NH₄-Form 1.

VT-XRPD analysis showing a loss in crystallinity coinciding with the mass loss (see FIG. 4).

Karl Fisher experiments were performed on the sample at about 150° C. and about 200° C. At the lower temperature the negligible amounts of water were observed, whereas at the higher temperature, about 0.5 eq (about 2%) water was detected. This data suggests that the ammonium salt may be a hemihydrate.

The sample became amorphous after 7 days at 25° C./97% RH but remained unchanged after 7 days at 40° C./75% RH. However, a possible form change was observed for the screening sample stored at 40° C./75% RH for 23 days. FIG. 5 shows XRPD diffractograms reflecting stability of obeticholic acid monoammonium salt post 7 days of storage under elevated conditions.

By GVS, the sample is slightly hygroscopic, with the water uptake over the 0-90% RH range approximately 1.5% (FIGS. 6 and 7). The form recovered after the experiment was unchanged by XRPD as shown in FIG. 8.

### Example 3. Cocrystal Screening Procedures

### Solvent Drop Grinding

A mixture of obeticholic acid (about 30 mg) and coformer (about 1.1 eq) was placed in a 2 mL stainless steel grinding jar with one 7 mm grinding ball. The materials were wetted with acetonitrile (about 10 μL), nitromethane (about 20 μL) or heptane (about 10 μL) and ground for about 1 h at 30 Hz using a Retsch Mixer Miller MM300. Majority of the samples initially ground with acetonitrile or nitromethane were dried, wetted with about 10 μL of n-heptane and ground for about 1 hour using the above conditions. All samples were then analysed by XRPD.

### Experiments Using THF/Heptane

An aliquot (about 0.19 mL) of a stock solution of obeticholic acid in THF (about 156 mg/mL) was added to neat coformer solid (about 1.1 eq). The samples were heated at about 50° C. for about 30 minutes and cooled to about 5° C. at about 0.1° C./minute. Stir speed of about 300 rpm was maintained throughout the experiments. Solutions obtained after cooling were treated with about 1 mL of heptane. All samples were then stirred at about 25-50° C. (8 h cycle) for about 7 days, then left to stand at ambient conditions for up to 5 days. Solids obtained were analysed by XRPD.

### Experiments Using Acetonitrile

An aliquot (1.4 mL) of a stock solution of obeticholic acid in acetonitrile (17 mg/mL) was added to neat coformer solid (1.1 eq). The samples were heated at 50° C. for 30 minutes and cooled to 5° C. at 0.1° C./minute. Stir speed of about 300 rpm was maintained throughout the experiments. Some samples were filtered after the cooling regime. The remaining of samples were stirred at about 25-50° C. (8 h cycle) for 5 days prior to filtration. Solids obtained were analysed by XRPD.

### Example 4. Obetichloic Acid-Ursodeoxycholic Acid Cocrystal—Form 1

Obeticholic acid (603 mg) and ursodeoxycholic acid (1.1 eq; 619 mg) were suspended in 28 mL of acetonitrile at 60° C. The sample was left stirring at 60° C. for 41 hours at a stirring rate of 300 rpm. The sample was filtered under suction filtration (partial vacuum) and dried under vacuum at 35° C. for 20 hours to afford 1012.2 mg of product. The mother liquor (filtrate) from the experiment was left to evaporate to dryness at 25° C. The obtained crystals were initially analysed by SCXRD.

### Characterisation of Obetichloic Acid-Ursodeoxycholic Acid Cocrystal

The NMR data (see FIG. 11) and the sharp melt by DSC (see FIG. 12) suggest this form is a cocrystal. This material was scaled up and characterised.

SCXRD analysis of OCA:UDCA cocrystal confirmed the solid phase purity batch (FIG. 17), with the experimental and simulated and XRPD diffractograms showing a good match (see FIG. 16). This pure cocrystal has a 2:1 ratio of OCA:UDCA as observed by NMR and SCXRD. The form displays no change after GVS experiment and showed a water uptake of ca. 1% w/w over the 0-90% RH range as shown in FIGS. 13-15. A DSC endotherm attributable to a melt is observed at ca. 174° C., with no significant weight loss observed by TGA prior to this temperature.

An anhydrous cocrystal of obeticholic acid with ursodeoxycholic acid (UDCA displays good solid form properties, possessing a good thermal profile up to a melt at ca. 174° C. and stability after exposure to high humidity. The diffraction peaks attributable to the cocrystal were unchanged by storage at 40° C./75% RH and 25° C./97% RH, indicating good stability of this phase (FIGS. 15 and 18).

TABLE 3
Solid state characterisation of UDCA cocrystal samples

| | | |
|---|---|---|
| XRPD | Crystalline | Crystalline |
| | (mixture of cocrystal + | (cocrystal) |
| | UDCA) | |
| ¹H-NMR | Consistent with ~1eq | Consistent with ~1eq |
| | OCA + 1eq UDCA | OCA + 0.5eq |
| | | UDCA + ≤0.03 |
| | | eq acetonitrile |
| TGA | No significant weight loss | No significant weight loss |
| | prior to sample melt, | prior to sample melt, |
| | Degradation starts at | Degradation starts at |
| | 200° C. | 200° C. |
| DSC | Endotherm | Endotherm |
| | (173.7° C., −82 J/g) | (173.5° C., −73 J/g) |
| Storage at | Unchanged by XRPD | Not determined |
| 40° C./75% | | |
| RH for 7 days | | |
| Storage at | Unchanged by XRPD | Not determined |
| 25° C./97% | | |
| RH for 7 days | | |
| GVS | 0.4% water uptake | 1% water uptake |
| | between 0 and 90% RH | between 0 and 90% RH |
| XRPD post | Unchanged by XRPD | Unchanged by XRPD |
| GVS | | |
| PLM | Irregular particles <40 | Irregular particles <40 |
| | μm in lenght | μm in lenght |
| Stoichiometry | ~1:1 | ~1:0.5 or 2:1 |
| OCA:coformer | (¹H-NMR) | (¹H-NMR) |

### Example 5. Single Crystal Experiment: OCA-UDCA (2:1) Cocrystal

Crystals obtained by slow evaporation in acetonitrile were evaluated in single crystal X-ray diffraction studies. A summary of all structural data for the obeticholic acid—ursodeoxycholic acid (2:1) cocrystal can be found in Table 4 and Table 5. The cocrystal crystallises in the monoclinic system, space group C2 with the final R1 [I>2σ(I)]=6.78%.

The single crystal structure confirmed the stoichiometry of the 2:1 cocrystal as indicated by the ¹H NMR analysis of the sample (FIG. 11).

Examination of the bond lengths supports the conclusion that the structure is a cocrystal. The asymmetric unit contains two molecules of obeticholic acid and one molecule of Ursodeoxycholic acid (FIG. 17), where anisotropic atomic displacement ellipsoids for the nonhydrogen atoms are shown at the 50% probability level (hydrogen atoms are displayed with an arbitrarily small radius).

FIG. 16 shows the experimental and calculated XRPD patterns of obeticholic acid—ursodeoxycholic acid (2:1) cocrystal. Slight differences between the XRPD patterns are attributable to lattice variations with temperature and preferred orientation.

TABLE 4
Sample details and crystal data for obeticholic
acid - ursodeoxycholic acid (2:1) cocrystal

| Compound | Obeticholic acid-ursodeoxycholic acid (2:1) |
|---|---|
| | cocrystal |
| Crystallisation solvent | Acetonitrile |
| Crystallisation method | Slow evaporation |
| Empirical formula* | C₇₆H₁₂₈O₁₂ |
| Formula weight | 1233.78 |
| Temperature | 100(2) K |
| Wavelength | 1.54178 Å |
| Crystal size | 0.350 × 0.200 × 0.080 mm |
| Crystal habit | Colourless Block |
| Crystal system | Monoclinic |
| Space group | C2 |
| Unit cell dimensions | a = 24.1898(3) Å α = 90° |
| | b = 11.87932(15) Å β = 108.4284(14)° |
| | c = 25.5901(4) Å γ = 90° |
| Volume | 6976.43(17) Å³ |
| Z | 4 |
| Density (calculated) | 1.175 Mg/m³ |
| Absorption coefficient | 0.606 mm⁻¹ |
| F(000) | 2720 |

*The formula described here correspond to the idealised compound were all the hydrogen atoms were found.

TABLE 5
Data collection and structure refinement for obeticholic
acid - ursodeoxycholic acid (2:1) cocrystal

| | |
|---|---|
| Diffractometer | SuperNova, Dual, Cu at zero, |
| | Atlas |
| Radiation source | SuperNova (Cu) X-ray Source, |
| | CuKα |
| Data collection method | Omega scans |
| Theta range for data | 8.944 to 74.482° |
| collection | |
| Index ranges | −30 ≤ h ≤ 30, −13 ≤ k ≤ |
| | 14, −31 ≤ l ≤ 31 |
| Reflections collected | 78239 |
| Independent reflections | 13823 [R(int) = 0.0667] |
| Coverage of independent | 99.4% |
| reflections | |
| Variation in check reflections | N/A |
| Absorption correction | Semi-empirical from equivalents |
| Max. and min. transmission | 1.00000 and 0.60238 |
| Structure solution technique | Direct |
| Structure solution program | SHELXTL (Sheldrick, 2013) |
| Refinement technique | Full-matrix least-squares on F2 |
| Refinement program | SHELXTL (Sheldrick, 2013) |
| Function minimized | Σw(Fₒ² − F_{c}²)² |
| Data/restraints/parameters | 13823/1/830 |
| Goodness-of-fit on F² | 1.039 |
| Δ/σₘₐₓ | 0.000 |
| Final R indices | R1 = 0.0678, wR2 = 0.1841 |
| 13186 data; I > 2σ(I) | R1 = 0.0702, wR2 = 0.1882 |
| all data | |
| Weighting scheme | w = 1/[σ² (Fₒ²) + (0.1262P)² + |
| | 7.9361P] |
| Absolute structure parameter | 0.02(7) |
| Extinction coefficient | n/a |
| Largest diff. peak and hole | 2.094 and −0.540 eÅ⁻³ |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A cocrystalline form of obeticholic acid (OCA) and a co-former.

2. The cocrystalline form of claim 1, wherein the co-former is a bile acid or bile acid derivative.

3. The cocrystalline form of claim 1, wherein the bile acid is ursodeoxycholic acid (UDCA).

4. The cocrystalline form of claim 1, wherein the bile acid is chenodeoxycholic acid (CDCA).

5. The cocrystalline form of claim 3, wherein the ratio of obeticholic acid to ursodeoxycholic acid is 2:1.

6. The cocrystalline form of claim 3, wherein the ratio of obeticholic acid to ursodeoxycholic acid is 1:1.

7. The cocrystalline form of claim 3, wherein the ratio of obeticholic acid to ursodeoxycholic acid is 1:0.5.

8. The cocrystalline form of claim 3, characterized by a DSC having an endotherm with an onset at about 174° C.

9. The cocrystalline form of claim 5, characterized by having X-ray powder diffraction (XRPD) comprising peaks at approximately at 7.4, 13.8, 14.9, 16.7 and 17.8 degrees 2-theta using Cu Kα radiation.

10. The cocrystalline form of claim 9, characterized by having X-ray powder diffraction (XRPD) comprising peaks at approximately 9.5, 15.2, 17.7, 24.7 degrees 2-theta using Cu Kα radiation.

11. The cocrystalline form of claim 10, characterized by having X-ray powder diffraction (XRPD) further comprising peaks at approximately 3.6, 8.3, 8.7, 10.3, 10.9, 11.2, 11.9, and 12.8 degrees 2-theta using Cu Kα radiation.

12. The cocrystalline form of claim 11, characterized by having X-ray powder diffraction (XRPD) further comprising peaks at approximately 16.8, 16.9, 17.9, 19.3, 19.8, 20.4, 20.7, 21.0, 22.3, 22.7, 23.0, 23.3, and 24.3 degrees 2-theta using Cu Kα radiation.

13. A cocrystalline form characterized by having X-ray powder diffraction (XRPD) comprising peaks at approximately 3.6, 7.4, 8.3, 8.7, 9.5, 10.3, 10.9, 11.2, 11.9, 12.8, 13.8, 14.9, 15.2, 16.7, 16.8, 16.9, 17.7, 17.8, 17.9, 19.3, 19.8, 20.4, 20.7, 21.0, 22.3, 22.7, 23.0, 23.3, 24.3, 24.7 degrees 2-theta using Cu Kα radiation.

14. The cocrystalline form of claim 5, characterized by having a monoclinic crystal system with the following unit cell parameters: a=approximately 24.19 Å, b=approximately 11.88 Å, and c=approximately 25.59 Å.

15. The cocrystalline form of claim 1 characterized by stability on storage at 40° C./75% RH and 25° C./97% RH.

16. A pharmaceutical composition comprising the cocrystalline form of any one of claims 1-15, and a pharmaceutically acceptable diluent, excipient or carrier.

17. A method of treating or preventing an FXR-mediated disease or disorder in a subject in need thereof, comprising administering a therapeutically effective amount of the cocrystalline form of any one of claims 1-15.

18. A method of modulating FXR activity in a subject in need thereof, comprising administering a therapeutically effective amount of the cocrystalline form of any one of claims 1-15.

19. A method of preparing the cocrystalline form of claim 1, comprising:
(a) dissolving OCA and the co-former in a solvent to form a solution;
(b) optionally heating the resulted solution;
(c) cooling the solution and optionally applying an anti-solvent; and
(f) filtering the product from step (c) and drying the product under vacuum.

20. The method of claim 19, wherein the solvent is acetonitrile.

21. The method of claim 19, wherein the solvent is tetrahydrofuran and the anti-solvent is heptane.
